(19) United States
(12) Patent Application Publication
(10) Pub. No.: US 2025/0017931 A1
(43) Pub. Date: Jan. 16, 2025

(54) METHODS OF PERSONALIZING CANCER TREATMENT

(71) Applicant: PROCESSA PHARMACEUTICALS, INC.,, Hanover, MD (US)

(72) Inventors: David YOUNG, Hanover, MD (US); Sian BIGORA, Hanover, MD (US); Lorenzo LEONI, Lodrino (CH); Khalid ISLAM, Lugano (CH)

(73) Assignee: PROCESSA PHARMACEUTICALS, INC.,, Hanover, MD (US)

(21) Appl. No.: 18/771,171

(22) Filed: Jul. 12, 2024

Related U.S. Application Data
(60) Provisional application No. 63/513,514, filed on Jul. 13, 2023.

(51) Int. Cl. A61K 31/513; A61P 35/00; G01N 33/574

(52) U.S. Cl. CPC A61K 31/513; A61P 35/00; G01N 33/57484

(57) ABSTRACT

The present disclosure provides, among other things, personalized methods of administering PCS6422 and capecitabine in combination, personalized methods of cancer treatment, and methods of determining the frequency of (i) PCS6422 and capecitabine or (ii) capecitabine administration in a subject.

### CROSS REFERENCE TO RELATED APPLICATIONS

This application claims the benefit of and priority to U.S. Provisional Patent Application No. 63/513,514, filed on Jul. 13, 2023, the contents of which are incorporated herein by reference in its entirety.

### BACKGROUND

The following description of the background of the present technology is provided simply as an aid in understanding the present technology and is not admitted to describe or constitute prior art to the present technology.

Fluoropyrimidine anti-metabolites are important anti-cancer therapeutics and 5-fluorouracil (5-FU) is the most commonly used drug of this class. Capecitabine (XELODA®) is a commonly used oral fluoropyrimidine and is a prodrug of 5-FU, which is converted via three enzymatic steps. First, capecitabine is converted to 5′-deoxy-5-fluorocytidine (5′-DFCR) by carboxylesterase; then 5′-DFCR is converted to 5′-deoxy-5-fluoruridine (5′-DFUR) by cytidine deaminase; and lastly, 5′-DFUR is converted to 5-FU by thymidine phosphorylasc.

Previous clinical trials examining the toxicity profile of capecitabine when compared to 5-FU have suggested a dose-dependent improved side effect profile compared to 5-FU/leucovorin (LV), including decreased stomatitis, diarrhea, nausea, and neutropenic sepsis. Despite a potential improved side effect profile, treatment with capecitabine has substantially higher rates of hyperbilirubinemia and hand-foot syndrome (HFS) and is rapidly degraded to α-fluoro-β-alanine (FBAL) by dihydropyrimidine dehydrogenase (DPD), an enzyme that can catabolize over 80% of 5-FU administered.

DPD levels can be highly variable, both within an individual subject (e.g., due to circadian patterns and tissue-specific variations) and from subject to subject. Such variations have been shown to be responsible, at least in part, for the variability in pharmacokinetic (PK), toxicity, and clinical response observed in clinical studies with 5-FU. While most individuals have DPD activity within the normal distribution, a small percentage (about <5%) of the population has DPD activity significantly below the normal distribution (e.g., due to inactivating mutations in one or two of the genes encoding DPD), which can lead to life-threatening or fatal toxicities when patients are administered conventional doses of 5-FU or capecitabine. Therefore, inhibiting the catabolism of 5-FU by inactivating DPD can provide an alternative approach to prolonging systemic exposure to 5-FU, increasing exposure to active metabolites, reducing exposure to toxic metabolites, and/or improving effectiveness.

### SUMMARY OF THE INVENTION

The present disclosure provides, among other things, personalized methods of treating a cancer in a subject in need thereof as well as methods of determining frequency of (i) PCS6422 and capecitabine or (ii) capecitabine administration to a subject and methods of treating cancer comprising determining whether a therapeutic regimen should be adjusted based on a therapeutic index.

In one aspect, the present disclosure provides a method of determining frequency of PCS6422 and capecitabine administration to a subject comprising: (i) administering at least one dose of an effective amount of PCS6422 to the subject; (ii) about 12 to about 24 hours after administering the at least one dose of the effective amount of PCS6422, administering at least one dose of a therapeutically effective amount of capecitabine to the subject, wherein a blood sample obtained from the subject was measured for α-fluoro-β-alanine (FBAL) and 5-fluorouracil (5-FU); (iii) determining one or more of the measured 5-FU, the measured FBAL, and/or a ratio of measured FBAL to measured 5-FU (FBAL: 5-FU ratio); and (iv) after a drug-free period, repeating steps (i) and (ii) when at least one of the following conditions are met: the measured 5-FU was less than a certain 5-FU threshold, the measured FBAL is greater than or equal to a certain FBAL threshold, the FBAL: 5-FU ratio determined was greater than or equal to a certain FBAL: 5-FU ratio threshold, the measured 5-FU is zero, or the measured FBAL is zero.

In some embodiments, the therapeutically effective amount of capecitabine is administered to the subject about 12 hours after administering the effective amount of PCS6422.

In some embodiments, the therapeutically effective amount of capecitabine is administered to the subject about 24 hours after administering the effective amount of PCS6422.

In some embodiments, the therapeutically effective amount of capecitabine is administered to the subject at both of about 12 hours and about 24 hours after administering the effective amount of PCS6422.

In some embodiments, the blood sample obtained from the subject was obtained about 12 to about 24 hours after administering the therapeutically effective amount of capecitabine to the subject.

In some embodiments, FBAL and 5-FU were measured daily starting on the first day of capecitabine treatment.

In some embodiments, steps (i) and (ii) are repeated 1-7, 8 or 14 days after step (ii).

In one aspect, the present disclosure provides a method of determining frequency of capecitabine administration to a subject comprising: (i) administering at least one dose of an effective amount of PCS6422 to the subject; (ii) about 12 to about 24 hours after administering the at least one dose of the effective amount of PCS6422, administering at least one dose of a therapeutically effective amount of capecitabine to the subject, wherein a blood sample obtained from the subject was measured for α-fluoro-β-alanine (FBAL) and/or 5-fluorouracil (5-FU); (iii) determining one or more of the measured 5-FU, the measured FBAL, or a ratio of measured FBAL to measured 5-FU (FBAL: 5-FU ratio); and (iv) stopping administering an additional dose of the therapeutically effective amount of capecitabine to the subject when the measured 5-FU was less than a certain 5-FU threshold, the measured FBAL is greater than or equal to a certain FBAL threshold, or the FBAL: 5-FU ratio determined was greater than or equal to a certain FBAL: 5-FU ratio threshold.

In some embodiments, stopping administering an additional dose of the therapeutically effective amount of capecitabine to the subject is followed by a drug-free period.

In some embodiments, after the drug-free period, steps (i) and (ii) are repeated.

In one aspect, the present disclosure provides a method of treating cancer in a subject in need thereof comprising: (i) administering at least one dose of an effective amount of PCS6422 to the subject; (ii) administering at least one dose of a therapeutically effective amount of capecitabine to the subject about 12 to about 24 hours after administering the at least one dose of the effective amount of PCS6422; (iii) measuring metabolites comprising one or more of α-fluoro-β-alanine (FBAL), 5′-deoxy-5-fluorocytidine (5′-DFCR), doxifluridine (5′-DFUR), and 5-fluorouracil (5-FU) in a blood sample obtained from the subject; and (iv) determining whether the therapeutic regimen should be adjusted based on a therapeutic index comprising the metabolite measurements determined in step (iii).

### DETAILED DESCRIPTION

PCS6422 is a potent and irreversible inactivator of dihydropyrimidine dehydrogenase (DPD). Low amounts of PCS6422 can inactivate DPD and render it incapable of catabolizing 5-FU, including 5-FU generated from the 5-FU prodrug, capecitabine. DPD enzyme activity subsequently occurs if not all of the existing DPD enzyme is inhibited and/or when active DPD reappears over time in subjects as a result of, for example, de novo DPD enzyme synthesis. PCS6422 converts the route of 5-FU elimination from metabolism to renal excretion by inhibiting the catabolism of 5-FU by DPD, thereby prolonging the half-life from 10-20 minutes to several hours. PCS6422 also prevents the formation of 5-FU catabolites such as FBAL, which appears to cause side effects such as hand-foot-syndrome, cardiotoxicity, and neurotoxicity and potentially antagonizes the antitumor activity of 5-FU. However, identifying appropriate dosing and/or therapeutic regimens of PCS6422 and capecitabine to realize the full potential of the combination therapy remains a challenge.

To advance technologies related to PCS6422 and capecitabine dosing, treatment regimens, and/or treatment, it is important to determine personalized therapeutic regimens and/or methods for personalizing such regimens, at least in part to account for the variability of DPD levels, and the variability of DPD de novo formation, both within an individual subject and from subject to subject.

The present disclosure provides, among other things, personalized methods of administering PCS6422 and capecitabine in combination, personalized methods of cancer treatment, and methods of determining the frequency of (i) PCS6422 and capecitabine or (ii) capecitabine administration in a subject. Such personalized methods, which are described in greater detail herein, can improve the safety and/or efficacy of capecitabine regimens.

It is to be appreciated that certain aspects, modes, embodiments, variations and features of the present methods are described below in various levels of detail in order to provide a substantial understanding of the present technology.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

### Definitions

Unless defined otherwise, all technical and scientific terms used herein have the meaning commonly understood by a person skilled in the art to which this disclosure belongs. The following references provide one of skill with a general definition of many of the terms used in the present disclosure. As used herein, the following terms have the meanings ascribed to them below, unless specified otherwise. The terminology used herein is for the purpose of describing particular embodiments only and is not intended to be limiting of the disclosure.

As used herein, the singular form “a,” “an,” and “the” include singular and plural references unless the context clearly dictates otherwise. For example, the term “a cell” includes a single cell as well as a plurality of cells, including mixtures thereof.

As used herein, the term “administration” of an agent or composition to a subject includes any route of introducing or delivering the agent or composition to a subject to perform its intended function. Administration can be carried out by any suitable route, including, but not limited to, oral administration, intravenous administration, and other suitable routes as described herein. Administration can include self-administration and administration by another.

As used herein, the term “adverse event” or “AE”, refers to any undesirable medical occurrence in a subject that was administered an agent or agents. An adverse event can therefore be any unfavorable and/or unintended sign (e.g., an abnormal laboratory finding), symptom, and/or condition temporally associated with the administration of the agent(s), whether or not considered related to the use of the agent(s).

As used herein, the term “agent,” refers to an entity (e.g., for example, a lipid, metal, nucleic acid, polypeptide, polysaccharide, small molecule, etc., or complex, combination, mixture or system ([e.g., cell, tissue, organism] thereof), or phenomenon (e.g., heat, electric current or field, magnetic force or field, etc.). In appropriate circumstances, as will be clear from context to those skilled in the art, the term may be utilized to refer to an entity that is or comprises a cell or organism, or a fraction, extract, or component thereof (e.g., a nucleic acid). Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. For example, an agent may be utilized in isolated or pure form; an agent may be utilized in crude form.

As used herein, the term “approximately” or “about” means plus or minus 10% as well as the specified number. For example, “about 10” should be understood as both “10” and “9-11”.

As used herein, the term “combination therapy” refers to those situations in which two or more different pharmaceutical agents are administered in overlapping regimens or the same regimen so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered, for example, a few minutes apart, or a few hours apart, or a few days apart.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used herein the term “effective amount” refers to a quantity of an agent or composition sufficient to achieve a desired biological result (e.g., a desired degree of inhibition of DPD).

As used herein, the term “diagnostic information” or “information for use in diagnosis” is any information that is useful in determining whether a patient has a disease or condition and/or in classifying the disease or condition into a phenotypic category or any category having significance with regard to prognosis of the disease or condition, or likely response to treatment (either treatment in general or any particular treatment) of the disease or condition. Similarly, diagnosis refers to providing any type of diagnostic information, including, but not limited to, whether a subject is likely to have a disease or condition (such as cancer), state, staging or characteristic of the disease or condition as manifested in the subject, information related to the nature or classification of a tumor, information related to prognosis and/or information useful in selecting an appropriate treatment. Selection of treatment may include the choice of a particular therapeutic (e.g., chemotherapeutic) agent or other treatment modality such as surgery, radiation, etc., a choice about whether to withhold or deliver therapy, a choice relating to dosing regimen (e.g., frequency or level of one or more doses of a particular therapeutic agent or combination of therapeutic agents), etc.

As used herein, the term “dosing regimen” or “therapeutic regimen” refers to a set of unit doses that are administered individually to a subject, typically separated by periods of time. A given therapeutic agent can have a recommended dosing regimen, which may involve one or more doses. A dosing regimen can comprise a plurality of doses, each of which are separated from one another by a period of time. Such a period of time may be the same length or different lengths. A dosing regimen can comprise a plurality of doses and at least two different time periods separating individual doses. A dosing regimen can be correlated with a desired therapeutic outcome when administered to a subject and/or across a population of subjects. A “dosing regimen” or “therapeutic regimen” can also refer to method used to partially or completely alleviate, ameliorate, relieve, inhibit, prevent, delay onset of, reduce severity of and/or reduce incidence of one or more symptoms or features of a particular disease, disorder, and/or condition (e.g., cancer). It may include a treatment or series of treatments designed to achieve a particular effect, e.g., reduction or elimination of a detrimental condition or disease such as cancer. The treatment may include administration of one or more compounds (e.g., therapeutic agents) either simultaneously, sequentially or at different times, for the same or different amounts of time.

As used herein, the term “durable clinical benefit” (DCB), has its art-understood meaning, referring to a clinical benefit that lasts for a relevant period of time. In some embodiments, such a clinical benefit is or comprises reduction in tumor size, increase in progression free survival, increase in overall survival, decrease in overall tumor burden, decrease in the symptoms caused by tumor growth such as pain, organ failure, bleeding, damage to the skeletal system, and other related sequelae of metastatic cancer and combinations thereof. In some embodiments, the relevant period of time is at least 1 month, 2 months, 3 months, 4 months, 5 months, 6 months, 7 months, 8 months, 9 months, 10 months, 11 months, 1 year, 2 years, 3 years, 4 years, 5 years, or longer. In some particular embodiments, the relevant period of time is 6 months.

As used herein, the term “therapeutically effective amount” refers to a quantity of an agent or composition sufficient to achieve a beneficial or desired clinical result upon treatment. In the context of therapeutic applications, the amount of a therapeutic agent administered to the subject can depend on the type and severity of the disease or condition and on the characteristics of the individual, such as general health, age, sex, body weight, and/or tolerance to drugs. It can also depend on the degree, severity, and type of disease. The skilled artisan will be able to determine appropriate dosages depending on these and other factors. A therapeutically effective amount can be administered to a subject in one or more doses. In terms of treatment, a therapeutically effective amount is an amount that is sufficient to palliate, ameliorate, stabilize, reverse or slow the progression of the disease, or otherwise reduce the pathological consequences of the disease. The therapeutically effective amount is generally determined by the physician on a case-by-case basis and is within the skill of one in the art.

As used herein, the term “favorable response” refers to a reduction in frequency and/or intensity of one or more symptoms, such as, reduction in tumor burden, full or partial remission, and/or other improvement in disease pathophysiology. Symptoms are reduced when one or more symptoms of a particular disease, disorder or condition is reduced in magnitude (e.g., intensity, severity, etc.) and/or frequency. For purposes of clarity, a delay in the onset of a particular symptom is considered one form of reducing the frequency of that symptom. Many cancer patients with smaller tumors have no symptoms. It is not intended that the technologies of the present disclosure be limited only to cases where the symptoms are eliminated. The present disclosure specifically contemplates treatment such that one or more symptoms is/are reduced (and the condition of the subject is thereby “improved”), albeit not completely eliminated. A favorable response can be established when a particular therapeutic regimen shows a statistically significant effect when administered across a relevant population or in an individual subject. A particular therapeutic regimen can be determined to have a favorable response when its administration is correlated with a relevant desired effect.

As used herein, the terms “improved”, “increased”, or “reduced”, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., a subject treated with a different therapeutic regimen of PCS6422 and capecitabine), or in a different, comparable subject (e.g., in a comparable subject or system that differs from the subject or system of interest in presence of one or more indicators of a particular disease, disorder or condition of interest, or in prior exposure to a condition or agent, etc.) Comparative terms can refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

As used herein, the term “long term benefit” refers to a desirable clinical outcome, e.g., observed after administration of a particular treatment or therapy (e.g., combination therapy) of interest, that is maintained for a clinically relevant period of time. For example, a long term benefit of cancer therapy can be or comprise (i) no evidence of disease (e.g., upon radiographic assessment) and/or (ii) stable or decreased volume of disease. A clinically relevant period of time can be, for example, at least 1 month, at least 2 months, at least 3 months, at least 6 months, at least 1 year, at least 5 years.

As used herein, the term “mutation” refers to a permanent change in the DNA sequence that makes up a gene. Mutations can range in size from a single DNA building block (DNA base) to a large segment of a chromosome. Mutations can include, for example, missense mutations, frameshift mutations, duplications, insertions, nonsense mutation, deletions and repeat expansions. A missense mutation can be a change in one DNA base pair that results in the substitution of one amino acid for another in the protein made by a gene. A nonsense mutation can also be a change in one DNA base pair. Instead of substituting one amino acid for another, however, the altered DNA sequence prematurely signals the cell to stop building a protein. An insertion can change the number of DNA bases in a gene by adding a piece of DNA. A deletion can change the number of DNA bases by removing a piece of DNA. In some embodiments, small deletions may remove one or a few base pairs within a gene, while larger deletions can remove an entire gene or several neighboring genes. A duplication can consists of a piece of DNA that is abnormally copied one or more times. A frameshift mutation can occur when the addition or loss of DNA bases changes a gene's reading frame. A reading frame consists of groups of 3 bases that each code for one amino acid. A frameshift mutation can shift the grouping of these bases and changes the code for amino acids. In some embodiments, insertions, deletions, and duplications can all be frameshift mutations. A repeat expansion is another type of mutation. In some embodiments, nucleotide repeats are short DNA sequences that are repeated a number of times in a row. For example, a trinucleotide repeat is made up of 3-base-pair sequences, and a tetranucleotide repeat is made up of 4-base-pair sequences. In some embodiments, a repeat expansion is a mutation that increases the number of times that the short DNA sequence is repeated.

As used herein, the term “objective response” refers to size reduction of a cancerous mass by a defined amount. The cancerous mass can be a tumor. A confirmed objective response can be a response confirmed a period of time after treatment.

As used herein, the term “objective response rate” (ORR) has its art-understood meaning referring to the proportion of patients with tumor size reduction of a predefined amount and for a minimum time period. Response duration is usually measured from the time of initial response until documented tumor progression. ORR can, for example, involve the sum of partial responses plus complete responses.

As used herein, the term “personalized” refers to a particular therapeutic regimen adapted to the needs and/or special circumstances of a particular subject and/or subject in need thereof (e.g., pharmacokinetic parameters of a particular agent or agents in a particular subject).

As used herein, the term “progression free survival” refers to the length of time during and after the treatment of a disease (e.g., cancer), that a subject lives with the disease, but it does not worsen. Measuring the progression free survival can be utilized as an assessment of how well a treatment (e.g., a combination treatment) works.

As used herein, the term “reduce” or “decrease” means to alter negatively by at least about 5% including, but not limited to, alter negatively by about 5%, by about 10%, by about 25%, by about 30%, by about 50%, by about 75%, or by about 100%.

As used herein, the term “response” refers to an alteration in a subject's condition that occurs as a result of, or correlates with, treatment (e.g., combination treatment). A response can be or comprise a beneficial response. A beneficial response may include stabilization of the condition (e.g., prevention or delay of deterioration expected or typically observed to occur absent the treatment), amelioration (e.g., reduction in frequency and/or intensity) of one or more symptoms of the condition and/or associated with the condition), and/or improvements in prognosis. A “response” may refer to response of an organism (e.g., a subject, such as a human), an organ, a cell, and/or a clinical response.

As used herein, a “reference” entity, system, amount, set of conditions, etc., is one against which a test entity, system, amount, set of conditions, etc. is compared as described herein. For example, a “reference” therapeutic regimen can be a control therapeutic regimen, e.g., a therapeutic regimen not disclosed herein. A reference or control can be tested and/or determined simultaneously with the testing or determination of interest. A reference or control can be a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

As used herein, the term “sample” typically refers to a biological sample obtained or derived from a source of interest (e.g., a subject and/or a subject in need thereof). A source of interest may be, for example, an organism, such as an animal or human. A biological sample can be or comprises biological tissue or fluid. A biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; plasma; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. A biological sample can be or comprises cells obtained from an individual. A sample can be a “primary sample” obtained directly from a source of interest by any appropriate means. For example, a primary biological sample can be obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a “secondary sample”, e.g., a preparation that is obtained by processing (e.g., by removing one or more components of and/or by adding one or more agents to) a primary sample. Such a “secondary sample” may comprise, for example nucleic acids or proteins extracted from a sample or obtained by subjecting a primary sample to techniques such as amplification or reverse transcription of mRNA, isolation and/or purification of certain components, etc.

As used herein, the terms “subject”, “individual”, or “patient” are used interchangeably and refer to an individual organism, a vertebrate, or a mammal and may include humans, non-human primates, rodents, and the like (e.g., which is to be the recipient of a particular treatment). In preferred embodiments, the individual, patient or subject is a human.

As used herein, the term “treatment cycle” refers to a period of treatment followed by a period of rest (e.g., a drug-free period). A treatment cycle can include more than one period of rest. A treatment cycle can be repeated (e.g., on a regular schedule or on a variable schedule). For example, a treatment given for one week followed by one week of rest (e.g., a one week drug-free period) can be one treatment cycle.

As used herein, the term “therapeutic index” refers to a plurality of therapeutic-regimen determining factors that can be used in combination as an indicator of overall effectiveness of an agent (e.g., PCS644, capecitabine) and/or whether a therapeutic regimen and/or treatment cycle should be adjusted (e.g., modifying the number of additional doses of a therapeutic agent that should be administered relative to the planned number of additional doses, lengthening or shortening a drug-free period relative to the planned duration). A therapeutic index can take into account therapeutic regimen-determining factors such as, for example and without limitation, disease type and/or severity, diagnostic information, subject responsiveness to a therapeutic agent, and/or pharmacokinetic analyses (or “metabolite measurements”) described herein.

As used herein, the term “variant”, in the context of molecules, such as nucleic acids or polypeptides, refers to a molecule that shows significant structural identity with a reference molecule but differs structurally from the reference molecule, e.g., in the presence or absence or in the level of one or more chemical moieties as compared to the reference entity. A variant can also differ functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. For example, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). A variant polypeptide or nucleic acid can show an overall sequence identity with a reference polypeptide or nucleic acid that is at least 85%, 86%, 87%, 88%, 89%, 90%, 91%, 92%, 93%, 94%, 95%, 96%, 97%, or 99%. A variant polypeptide or nucleic acid can comprise about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference.

### Treatments

The present disclosure provides, among other things, personalized methods of treating a cancer in a subject in need thereof, comprising: (i) administering at least one dose of an effective amount of PCS6422 to the subject; (ii) about 12 to about 36 hours (such as about 12 to about 24 hours) after administering the at least one dose of the effective amount of PCS6422, administering at least one dose of a therapeutically effective amount of capecitabine to the subject; and (iii) optionally, after a drug-free period, repeating steps (i) and (ii).

In another aspect, the present disclosure provides methods of treating a cancer in a subject in need thereof comprising: (i) administering at least one dose of an effective amount of PCS6422 to the subject; and (ii) about 12 to about 36 hours (such as about 12 to about 24 hours) after administering the at least one dose of the effective amount of PCS6422, administering at least one dose of a therapeutically effective amount of capecitabine to the subject, wherein a blood sample obtained from the subject was measured for one or more of: α-fluoro-β-alanine (FBAL), 5′-deoxy-5-fluorocytidine (5′-DFCR), doxifluridine (5′-DFUR), capecitabine, and 5-fluorouracil (5-FU). Such methods of treating cancer can further comprise determining whether the therapeutic regimen should be adjusted based on a therapeutic index including the measurements of the one or more of: FBAL, 5-DFCR, 5′-DFUR, capecitabine, and 5-FU. Adjusting a therapeutic regimen can include, for example, modifying the number of additional doses of a therapeutic agent should be administered relative to the planned number of additional doses, lengthening or shortening a drug-free period relative to the planned duration, changing the number of treatment cycles to be administered relative to the planned number of treatment cycles.

In another aspect, the present disclosure provides methods of treating cancer in a subject in need thereof comprising: (i) administering at least one dose of an effective amount of PCS6422 to the subject; and (ii) administering at least one dose of a therapeutically effective amount of capecitabine to the subject about 12 to about 36 hours (such as about 12 to about 24 hours) after administering the at least one dose of the effective amount of PCS6422; (iii) measuring metabolites comprising one or more of α-fluoro-β-alanine (FBAL), 5′-deoxy-5-fluorocytidine (5′-DFCR), doxifluridine (5′-DFUR), and 5-fluorouracil (5-FU) in a blood sample obtained from the subject; and (iv) determining whether the therapeutic regimen should be adjusted based on a therapeutic index comprising the metabolite measurements determined in step (iii). In some embodiments, measuring metabolites of step (iii) comprises measuring one or more of FBAL and 5-FU. In some embodiments, such methods further comprise determining a FBAL: 5-FU ratio. Such metabolite measurements are also referred to as “pharmacokinetic analyses” as described elsewhere herein.

In another aspect, the present disclosure provides methods of determining frequency of PCS6422 and capecitabine administration to a subject comprising: (i) administering at least one dose of an effective amount of PCS6422 to the subject; (ii) about 12 to about 36 hours (such as about 12 to about 24 hours) after administering the at least one dose of the effective amount of PCS6422, administering at least one dose of a therapeutically effective amount of capecitabine to the subject, wherein a blood sample obtained from the subject was measured for α-fluoro-β-alanine (FBAL) and 5-fluorouracil (5-FU); (iii) determining one or more of the measured 5-FU, the measured FBAL, and/or a ratio of measured FBAL to measured 5-FU (FBAL: 5-FU ratio); and (iv) after a drug-free period, repeating steps (i) and (ii) when at least one of the following conditions are met: the measured 5-FU was less than a certain 5-FU threshold, the measured FBAL is greater than or equal to a certain FBAL threshold, the FBAL: 5-FU ratio determined was greater than or equal to a certain FBAL: 5-FU ratio threshold, the measured 5-FU is zero, or the measured FBAL is zero.

In yet another aspect, the present disclosure provides methods of determining frequency of capecitabine administration to a subject comprising (i) administering at least one dose of an effective amount of PCS6422 to the subject; (ii) about 12 to about 36 hours (such as about 12 to about 24 hours) after administering the at least one dose of the effective amount of PCS6422, administering at least one dose of a therapeutically effective amount of capecitabine to the subject, wherein a blood sample obtained from the subject was measured for α-fluoro-β-alanine (FBAL) and/or 5-fluorouracil (5-FU); (iii) determining one or more of the measured 5-FU, the measured FBAL, or a ratio of measured FBAL to measured 5-FU (FBAL: 5-FU ratio); and (iv) stopping administering an additional dose of the therapeutically effective amount of capecitabine to the subject when the measured 5-FU was less than a certain 5-FU threshold, the measured FBAL is greater than or equal to a certain FBAL threshold, or the FBAL: 5-FU ratio determined was greater than or equal to a certain FBAL: 5-FU ratio threshold.

In some embodiments, the therapeutically effective amount of capecitabine is administered to the subject about 12 hours after administering the effective amount of PCS6422. In some embodiments, the therapeutically effective amount of capecitabine is administered to the subject about 12 to about 36 hours after administering the effective amount of PCS6422. In some embodiments, the therapeutically effective amount of capecitabine is administered to the subject about 12 to about 24 hours after administering the effective amount of PCS6422. In some embodiments, the therapeutically effective amount of capecitabine is administered to the subject at both of about 12 hours and about 36 hours after administering the effective amount of PCS6422. In some embodiments, the therapeutically effective amount of capecitabine is administered to the subject at both of about 12 hours and about 24 hours after administering the effective amount of PCS6422.

Methods of the disclosure can comprise administering an additional dose of an effective amount of PCS6422. In some embodiments, 2, 3, 4, 5, or 6 doses of an effective amount of PCS6422 are administered. In some embodiments, 2 doses of an effective amount of PCS6422 are administered. In some embodiments, 3 doses of an effective amount of PCS6422 are administered. In some embodiments, 4 doses of an effective amount of PCS6422 are administered. In some embodiments, 5 doses of an effective amount of PCS6422 are administered. In some embodiments, 6 doses of an effective amount of PCS6422 are administered.

In some embodiments, methods of the present disclosure comprise administering the at least one dose of an effective amount of PCS6422 to the subject on Day 1 of a treatment cycle. In some embodiments, methods of the present disclosure comprise administering the at least one dose of an effective amount of PCS6422 to the subject on Day 1 and Day 5 of a treatment cycle. In some embodiments, methods of the present disclosure comprise administering the at least one dose of an effective amount of PCS6422 to the subject on Day 1 and Day 6 of a treatment cycle. In some embodiments, methods of the present disclosure comprise administering the at least one dose of an effective amount of PCS6422 to the subject on Day 1 and Day 8 of a treatment cycle. In some embodiments, methods of the present disclosure comprise administering the at least one dose of an effective amount of PCS6422 to the subject on Day 1, Day 8, and Day 15 of a treatment cycle.

Methods of the present disclosure can comprise administering a dose of an effective amount of PCS6422 as a divided dose.

Methods of the disclosure can comprise administering an additional dose of a therapeutically effective amount of capecitabine to the subject about 12 to about 36 hours (such as about 12 to about 24 hours) after administering a previous dose of a therapeutically effective amount of capecitabine to the subject. In some embodiments, 2, 3, 4, 5, 6, 7, 8, 9, 10, 11, 12, 13, 14, 15, or 16 doses of a therapeutically effective amount of capecitabine are administered. In some embodiments, 2 doses of a therapeutically effective amount of capecitabine are administered. In some embodiments, 3 doses of a therapeutically effective amount of capecitabine are administered. In some embodiments, 4 doses of a therapeutically effective amount of capecitabine are administered. In some embodiments, 5 doses of a therapeutically effective amount of capecitabine are administered. In some embodiments, 6 doses of a therapeutically effective amount of capecitabine are administered. In some embodiments, 7 doses of a therapeutically effective amount of capecitabine are administered. In some embodiments, 8 doses of a therapeutically effective amount of capecitabine are administered. In some embodiments, 9 doses of a therapeutically effective amount of capecitabine are administered. In some embodiments, 10 doses of a therapeutically effective amount of capecitabine are administered. In some embodiments, 11 doses of a therapeutically effective amount of capecitabine are administered. In some embodiments, 12 doses of a therapeutically effective amount of capecitabine are administered. In some embodiments, 13 doses of a therapeutically effective amount of capecitabine are administered. In some embodiments, 14 doses of a therapeutically effective amount of capecitabine are administered. In some embodiments, 15 doses of a therapeutically effective amount of capecitabine are administered. In some embodiments, 16 doses of a therapeutically effective amount of capecitabine are administered.

In some embodiments, methods of the disclosure can comprise stopping administering an additional dose of the therapeutically effective amount of capecitabine to the subject when the measured 5-FU was less than a certain 5-FU threshold, the measured FBAL is greater than or equal to a certain FBAL threshold, or the FBAL: 5-FU ratio determined was greater than or equal to a certain FBAL: 5-FU ratio threshold. In some such embodiments, stopping administering an additional dose of the therapeutically effective amount of capecitabine to the subject is followed by a drug-free period (e.g., as described herein). In some such embodiments, after the drug-free period, steps (i) and (ii) are repeated.

In some embodiments, method of the disclosure can comprise adjusting a therapeutic regimen based on a therapeutic index comprising the metabolite measurements (“pharmacokinetic analysis) described herein. Adjusting a therapeutic regimen can include, for example, modifying the number of additional doses of a therapeutic agent should be administered relative to the planned number of additional doses, lengthening or shortening a drug-free period relative to the planned duration, changing the number of treatment cycles to be administered relative to the planned number of treatment cycles.

In some embodiments, methods of the present disclosure comprise administering the at least one dose of a therapeutically effective amount of capecitabine to the subject on each of Days 2-3, Days 2-4, Days 2-5, Days 2-6, Days 2-7, Days 2-8, Days 2-10, Days 2-12, Days-15, Days 3-4, Days 3-5, Days 3-6, Days 3-7, Days 3-8, Days 4-5, Days 4-6, Days 4-7, Days 4-8, Days 5-6, Days 5-7, Days 5-8, Days 6-7, Days 6-8, or Days 7-8 of a treatment cycle or any combination thereof. In some embodiments, methods of the present disclosure comprise administering the at least one dose of a therapeutically effective amount of capecitabine to the subject on each of Days 2-3 of a treatment cycle. In some embodiments, methods of the present disclosure comprise administering the at least one dose of a therapeutically effective amount of capecitabine to the subject on each of Days 2-4 of a treatment cycle. In some embodiments, methods of the present disclosure comprise administering the at least one dose of a therapeutically effective amount of capecitabine to the subject on each of Days 2-5 of a treatment cycle. In some embodiments, methods of the present disclosure comprise administering the at least one dose of a therapeutically effective amount of capecitabine to the subject on each of Days 2-6 of a treatment cycle. In some embodiments, methods of the present disclosure comprise administering the at least one dose of a therapeutically effective amount of capecitabine to the subject on each of Days 2-7 of a treatment cycle. In some embodiments, methods of the present disclosure comprise administering the at least one dose of a therapeutically effective amount of capecitabine to the subject on each of Days 2-8 of a treatment cycle. In some embodiments, methods of the present disclosure comprise administering the at least one dose of a therapeutically effective amount of capecitabine to the subject on each of Days 2-10 of a treatment cycle. In some embodiments, methods of the present disclosure comprise administering the at least one dose of a therapeutically effective amount of capecitabine to the subject on each of Days 2-12 of a treatment cycle. In some embodiments, methods of the present disclosure comprise administering the at least one dose of a therapeutically effective amount of capecitabine to the subject on each of Days 2-15 of a treatment cycle. In some embodiments, methods of the present disclosure comprise administering the at least one dose of a therapeutically effective amount of capecitabine to the subject on each of Days 3-4 of a treatment cycle. In some embodiments, methods of the present disclosure comprise administering the at least one dose of a therapeutically effective amount of capecitabine to the subject on each of Days 3-5 of a treatment cycle. In some embodiments, methods of the present disclosure comprise administering the at least one dose of a therapeutically effective amount of capecitabine to the subject on each of Days 3-6 of a treatment cycle. In some embodiments, methods of the present disclosure comprise administering the at least one dose of a therapeutically effective amount of capecitabine to the subject on each of Days 3-7 of a treatment cycle. In some embodiments, methods of the present disclosure comprise administering the at least one dose of a therapeutically effective amount of capecitabine to the subject on each of Days 3-8 of a treatment cycle. In some embodiments, methods of the present disclosure comprise administering the at least one dose of a therapeutically effective amount of capecitabine to the subject on each of Days 4-5 of a treatment cycle. In some embodiments, methods of the present disclosure comprise administering the at least one dose of a therapeutically effective amount of capecitabine to the subject on each of Days 4-6 of a treatment cycle. In some embodiments, methods of the present disclosure comprise administering the at least one dose of a therapeutically effective amount of capecitabine to the subject on each of Days 4-7 of a treatment cycle. In some embodiments, methods of the present disclosure comprise administering the at least one dose of a therapeutically effective amount of capecitabine to the subject on each of Days 4-8 of a treatment cycle. In some embodiments, methods of the present disclosure comprise administering the at least one dose of a therapeutically effective amount of capecitabine to the subject on each of Days 5-6 of a treatment cycle. In some embodiments, methods of the present disclosure comprise administering the at least one dose of a therapeutically effective amount of capecitabine to the subject on each of Days 5-7 of a treatment cycle. In some embodiments, methods of the present disclosure comprise administering the at least one dose of a therapeutically effective amount of capecitabine to the subject on each of Days 5-8 of a treatment cycle. In some embodiments, methods of the present disclosure comprise administering the at least one dose of a therapeutically effective amount of capecitabine to the subject on each of Days 6-7 of a treatment cycle. In some embodiments, methods of the present disclosure comprise administering the at least one dose of a therapeutically effective amount of capecitabine to the subject on each of Days 6-8 of a treatment cycle. In some embodiments, methods of the present disclosure comprise administering the at least one dose of a therapeutically effective amount of capecitabine to the subject on each of Days 7-8 of a treatment cycle. In some embodiments, methods of the present disclosure comprise administering the at least one dose of a therapeutically effective amount of capecitabine to the subject on each of Days 2-3, 9-10, and 16-17 of a treatment cycle.

In some embodiments, methods of the present disclosure comprise administering the at least one dose of a therapeutically effective amount of capecitabine to the subject on any or each of Days 2-8 of a treatment cycle, wherein at least one drug-free period is included within Days 3-7 of the treatment cycle.

In some embodiments, methods of the present disclosure comprise administering the at least one dose of a therapeutically effective amount of capecitabine to the subject on Day 2 of a treatment cycle. In some embodiments, methods of the present disclosure comprise administering the at least one dose of a therapeutically effective amount of capecitabine to the subject on Day 3 of a treatment cycle. In some embodiments, methods of the present disclosure comprise administering the at least one dose of a therapeutically effective amount of capecitabine to the subject on Day 4 of a treatment cycle. In some embodiments, methods of the present disclosure comprise administering the at least one dose of a therapeutically effective amount of capecitabine to the subject on Day 5 of a treatment cycle. In some embodiments, methods of the present disclosure comprise administering the at least one dose of a therapeutically effective amount of capecitabine to the subject on Day 6 of a treatment cycle. In some embodiments, methods of the present disclosure comprise administering the at least one dose of a therapeutically effective amount of capecitabine to the subject on Day 7 of a treatment cycle. In some embodiments, methods of the present disclosure comprise administering the at least one dose of a therapeutically effective amount of capecitabine to the subject on Day 8 of a treatment cycle. In some embodiments, methods of the present disclosure comprise administering the at least one dose of a therapeutically effective amount of capecitabine to the subject on Day 9 of a treatment cycle. In some embodiments, methods of the present disclosure comprise administering the at least one dose of a therapeutically effective amount of capecitabine to the subject on Day 10 of a treatment cycle. In some embodiments, methods of the present disclosure comprise administering the at least one dose of a therapeutically effective amount of capecitabine to the subject on Day 11 of a treatment cycle. In some embodiments, methods of the present disclosure comprise administering the at least one dose of a therapeutically effective amount of capecitabine to the subject on Day 12 of a treatment cycle. In some embodiments, methods of the present disclosure comprise administering the at least one dose of a therapeutically effective amount of capecitabine to the subject on Day 13 of a treatment cycle. In some embodiments, methods of the present disclosure comprise administering the at least one dose of a therapeutically effective amount of capecitabine to the subject on Day 14 of a treatment cycle. In some embodiments, methods of the present disclosure comprise administering the at least one dose of a therapeutically effective amount of capecitabine to the subject on Day 15 of a treatment cycle. In some embodiments, methods of the present disclosure comprise administering the at least one dose of a therapeutically effective amount of capecitabine to the subject on Day 16 of a treatment cycle. In some embodiments, methods of the present disclosure comprise administering the at least one dose of a therapeutically effective amount of capecitabine to the subject on Day 17 of a treatment cycle.

Methods of the present disclosure can comprise administering a dose of a therapeutically effective amount of capecitabine as a divided dose.

### Drug-Free Period

Methods and/or treatment cycles of the present disclosure can include one or more drug-free periods (e.g., after administration of at least one dose of a therapeutically effective amount of capecitabine to the subject and/or subject in need thereof). A drug-free period refers to a time period in which a subject and/or subject in need thereof (e.g., a subject with cancer) is not administered with any doses of either of PCS6422 or capecitabine. Alternatively or additionally, a drug-free period can refer to a time period in which a subject and/or a subject in need thereof (e.g., a subject with cancer) is determined to have no or a non-detectable amount of PCS6422 and/or capecitabine in a blood sample obtained from the subject using pharmacokinetic analyses as described herein.

In some embodiments, the drug-free period is about 1 to about 28 days, about 1 to about 26 days, about 1 to about 24 days, about 1 to about 22 days, about 1 to about 21 days, about 1 to about 15 days, about 1 to about 14 days, about 1 to about 12 days, about 1 to about 10 days, about 1 to about 8 days, about 1 to about 6 days, about 1 to about 4 days, about 1 to about 3 days, or about 1 to about 2 days. In some embodiments, the drug free period is about 1 to about 2 days. In some embodiments, the drug free period is about 1 to about 3 days. In some embodiments, the drug free period is about 1 to about 4 days. In some embodiments, the drug free period is about 1 to about 5 days. In some embodiments, the drug free period is about 1 to about 6 days. In some embodiments, the drug free period is about 1 to about 7 days. In some embodiments, the drug free period is about 1 to about 8 days. In some embodiments, the drug free period is about 1 to about 10 days. In some embodiments, the drug free period is about 1 to about 12 days. In some embodiments, the drug free period is about 1 to about 14 days. In some embodiments, the drug free period is about 1 to about 15 days. In some embodiments, the drug free period is about 1 to about 21 days. In some embodiments, the drug free period is about 1 to about 22 days. In some embodiments, the drug free period is about 1 to about 24 days. In some embodiments, the drug free period is about 1 to about 26 days. In some embodiments, the drug free period is about 1 to about 28 days.

In some embodiments, the drug-free period is about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, 21 days, 22 days, 24 days, 26 days, or 28 days. In some embodiments, the drug-free period is about 1 day. In some embodiments, the drug-free period is about 2 days. In some embodiments, the drug-free period is about 3 days. In some embodiments, the drug-free period is about 4 days. In some embodiments, the drug-free period is about 5 days. In some embodiments, the drug-free period is about 6 days. In some embodiments, the drug-free period is about 7 days. In some embodiments, the drug-free period is about 8 days. In some embodiments, the drug-free period is about 10 days. In some embodiments, the drug-free period is about 12 days. In some embodiments, the drug-free period is about 14 days. In some embodiments, the drug-free period is about 15 days. In some embodiments, the drug-free period is about 21 days. In some embodiments, the drug-free period is about 22 days. In some embodiments, the drug-free period is about 24 days. In some embodiments, the drug-free period is about 26 days. In some embodiments, the drug-free period is about 28 days.

In some embodiments, the drug-free period occurs on Day 3 of a treatment cycle. In some embodiments, the drug-free period occurs on Day 4 of a treatment cycle. In some embodiments, the drug-free period occurs on Day 5 of a treatment cycle. In some embodiments, the drug-free period occurs on Day 6 of a treatment cycle. In some embodiments, the drug-free period occurs on Day 7 of a treatment cycle. In some embodiments, the drug-free period occurs on Days 3-7 of a treatment cycle. In some embodiments, the drug-free period occurs on Days 4-7 of a treatment cycle. In some embodiments, the drug-free period occurs on Days 3-5 of a treatment cycle. In some embodiments, the drug-free period occurs on Days 4-5 of a treatment cycle. In some embodiments, the drug-free period occurs on Days 5-7 of a treatment cycle. In some embodiments, the drug-free period occurs on Days 5-14 of a treatment cycle. In some embodiments, the drug-free period occurs on Days 7-14 of a treatment cycle. In some embodiments, the drug-free period occurs on Days 9-14 of a treatment cycle. In some embodiments, the drug-free period occurs on Days 16-21 of a treatment cycle.

A drug-free period of the present disclosure can be a split drug-free period in a given treatment cycle (e.g., the total number of drug-free days in a given treatment cycle are divided throughout the course of a treatment cycle). For example and without limitation, PCS6422 may be administered on Day 1 and a therapeutically effective amount of capecitabine is administered on each of Days 2 and 3, followed by a drug-free period on Days 4-5. Then, on Day 6, a second dose of an effective amount of PCS6422 is administered and a therapeutically effective amount of capecitabine is administered on each of Days 7-8, followed by the remaining portion of drug free-period in the treatment cycle on days 9-14. In some embodiments, the drug-free period occurs on Days 4-7, 11-14, and 18-28 of a treatment cycle.

A drug-free period of the present disclosure can be a variable drug-free period (e.g., the drug free period of a first treatment cycle is a different length than the drug-free period of a second treatment cycle). For example and without limitation, a first treatment cycle may include a drug-free period of 3 days (e.g., days 3, 4, and 5 of the first treatment cycle) and a subsequently second treatment cycle may include a drug-free period of 5 days (e.g., days 3, 4, 5, 6, and 7 of the second treatment cycle). A drug-free period may be adjusted from one treatment cycle to the next for a plurality of reasons, including, for example, the observation of adverse side effects in the subject and/or the determined measurements of 5-FU, the measurements of FBAL, and/or a ratio of measured FBAL to measured 5-FU (FBAL: 5-FU ratio) in a blood sample obtained from a subject and/or a subject in need thereof (e.g., as described elsewhere herein).

### PCS6422

PCS6422 (chemical name 5-ethynyl-1H-pyrimidine-2, 4-dione and previously known as eniluracil, 5-ethynyluracil, 776C85, and ADH300004) is a potent and irreversible inactivator of DPD. Without wishing to be bound by any one theory, PCS6422 is understood to convert the route of 5-FU elimination from metabolism to renal excretion and increases the 5-FU elimination half-life from 10-20 minutes to several hours. PCS6422 also prevents the formation of 5-FU catabolites such as FBAL, which is understood to cause neurotoxicity and potentially antagonize the antitumor activity of 5-FU. FBAL has also been demonstrated to decrease the anti-tumor activity of 5-FU in laboratory animals. Furthermore, because DPD is present at variable levels (e.g., with a given subject and between subjects), the highly variable and non-linear pharmacokinetics (PK) of 5-FU can demonstrate increased predictability and/or linearity when DPD is inactivated by PCS6422 in cancer patients. PCS6422 is generally administered orally. In some embodiments, PCS6422 is administered orally. In some embodiments, PCS6422 is administered intravenously.

PCS6422 can be administered in accordance with the present disclosure in an effective amount. In some embodiments, an effective amount of PCS6422 is the maximum tolerated dose. In some embodiments, an effective amount of PCS6422 is about 10 mg to about 60 mg, about 15 mg to about 60 mg, about 20 mg to about 60 mg, about 25 mg to about 50 mg, or about 30 mg to about 50 mg. In some embodiments, an effective amount of PCS6422 is about 10 mg, about 15 mg, about 20 mg, about 25 mg, about 30 mg, about 35 mg, about 40 mg, about 45 mg, about 50 mg, about 55 mg, or about 60 mg. In some embodiments, an effective amount of PCS6422 is about 10 mg. In some embodiments, an effective amount of PCS6422 is about 20 mg. In some embodiments, an effective amount of PCS6422 is about 30 mg. In some embodiments, an effective amount of PCS6422 is about 40 mg. In some embodiments, an effective amount of PCS6422 is about 50 mg. In some embodiments, an effective amount of PCS6422 is about 60 mg.

An effective amount of PCS6422 can be administered in accordance with the methods of the present disclosure in a divided dose (e.g., as a fraction of a full dose). In some embodiments, a divided dose (e.g., a half dose) of an effective amount of PCS6422 is administered in the morning, followed by the remaining fraction of the divided dose (e.g., half dose) in the evening. In some embodiments, a divided dose of an effective amount of PCS6422 is administered at time 0 on Day 1 of a treatment cycle, followed by the remaining fraction of the divided dose at time about 8-16 hours on Day 1 of the treatment cycle. In some embodiments, the effective amount of PCS6422 of 40 mg and is administered as a divided dose of 20 mg two times per day.

### Capecitabine

Capecitabine (Xeloda®) is a commonly used oral fluoropyrimidine and is a prodrug of 5-FU which is converted via three enzymatic steps. First, capecitabine is converted to 5′-deoxy-5-fluorocytidine (5′-DFCR) by carboxylesterase; then 5′-DFCR is converted to 5′-deoxy-5-fluorouridine (5′-DFUR) by cytidine deaminase; and finally, 5′-DFUR is converted to 5-FU by thymidine phosphorylase. Capecitabine is generally administered orally. In some embodiments, capecitabine is administered orally. In some embodiments, capecitabine is administered intravenously.

Capecitabine can be administered in accordance with the present disclosure in a therapeutically effective amount. In some embodiments, a therapeutically effective amount of capecitabine is the maximum tolerated dose. In some embodiments, a therapeutically effective amount of capecitabine is about 25 mg to about 450 mg, about 25 mg to about 300 mg, about 25 mg to about 225 mg, about 25 mg to about 150 mg, about 25 mg to about 75 mg, about 50 mg to about 450 mg, about 50 mg to about 300 mg, about 50 mg to about 225 mg, about 50 mg to about 150 mg, about 75 mg to about 450 mg, about 75 mg to about 300 mg, about 75 mg to about 225 mg, or about 75 mg to about 150 mg. In some embodiments, a therapeutically effective amount of capecitabine is about 25 mg, about 50 mg, about 75 mg, about 150 mg, about 225 mg, about 300 mg, or about 450 mg. In some embodiments, a therapeutically effective amount of capecitabine is about 25 mg. In some embodiments, a therapeutically effective amount of capecitabine is about 50 mg. In some embodiments, a therapeutically effective amount of capecitabine is about 75 mg. In some embodiments, a therapeutically effective amount of capecitabine is about 150 mg. In some embodiments, a therapeutically effective amount of capecitabine is about 225 mg. In some embodiments, a therapeutically effective amount of capecitabine is about 300 mg. In some embodiments, a therapeutically effective amount of capecitabine is about 450 mg.

A therapeutically effective amount of capecitabine can be administered in accordance with the methods of the present disclosure in a divided dose (e.g., as a fraction of a full dose). In some embodiments, a divided dose (e.g., a half dose) of a therapeutically effective amount of capecitabine is administered in the morning, followed by the remaining fraction of the divided dose (e.g., half dose) in the evening. For example, a therapeutically effective amount of capecitabine can be about 450 mg and can be administered as a divided dose of 225 mg two times per day.

### Pharmacokinetic Analyses

Pharmacokinetic analyses can be utilized to evaluate capecitabine and its metabolites (e.g., to personalize methods described herein). Without wishing to be bound by any one theory, pharmacokinetic analyses of one or more of the measured 5-FU, the measured FBAL, and/or a ratio of measured FBAL to measured 5-FU (FBAL: 5-FU ratio) can be indicative of when enough DPD has formed (e.g., by de novo synthesis) to significantly decrease 5-FU potency and/or efficacy (e.g., to a similar level of capecitabine when administered alone), thus informing the appropriate time to administer additional doses of an effective amount of PCS6422 and/or a therapeutically effective amount of capecitabine (e.g., including additional treatment cycles) and/or indicating whether subject and/or a subject in need thereof (e.g., a subject with cancer) has no or a non-detectable amount of PCS6422 and/or capecitabine in a blood sample obtained from the subject. Alternatively, and without wishing to be bound by any one theory, pharmacokinetic analyses of one or more of the measured 5-FU, the measured FBAL, or the FBAL: 5-FU ratio can be indicative of when only low amounts of additional capecitabine administered will be metabolized to 5-FU (e.g., due to the presence of excess drug in the subject and/or subject in need thereof, e.g., due to inhibition of DPD by PCS6422) and rather, could potentially contribute to adverse events (e.g., due to capecitabine catabolites), thus informing when to stop administering additional doses of a therapeutically effective amount of capecitabine to a subject and/or a subject in need thereof. Accordingly, such analyses can be utilized to personalize the frequency of (i) PCS6422 and capecitabine or (ii) capecitabine in a therapeutic regimen and/or the treatment cycles for a particular subject and/or subject in need thereof. Thus, in some embodiments, a treatment cycle of a therapeutic regimen of the disclosure can be repeated when at least one of the following conditions are met: the measured 5-FU was less than a certain 5-FU threshold, the measured FBAL is greater than or equal to a certain FBAL threshold, the FBAL: 5-FU ratio determined was greater than or equal to a certain FBAL: 5-FU ratio threshold, the measured 5-FU is zero, or the measured FBAL is zero, whereas, in some embodiments, a treatment cycle or a therapeutic regimen of the disclosure comprises stopping administering an additional dose of the therapeutically effective amount of capecitabine to the subject when the measured 5-FU was less than a certain 5-FU threshold, the measured FBAL is greater than or equal to a certain FBAL threshold, or the FBAL: 5-FU ratio determined was greater than or equal to a certain FBAL: 5-FU ratio threshold.

Pharmacokinetic analyses of the present disclosure may be used in combination with other therapeutic regimen-determining factors to adjust a therapeutic regimen and/or treatment cycle of the present disclosure (e.g., personalize such a method). Such other therapeutic regimen-determining factors can include, for example, disease type and/or severity and subject responsiveness to a therapeutic agent. Such factors in combination with the pharmacokinetic analysis of the present disclosure (e.g., “metabolite measurements”) may be evaluated (e.g., by a health-care practitioner) to determine a therapeutic index which can be used as a basis to adjust a therapeutic regimen (e.g., of a treatment cycle) of the present disclosure.

A plurality of analytical methods are available and known to those of ordinary skill in the art and can be utilized to measure the concentrations of one or more of: α-fluoro-β-alanine (FBAL), 5′-deoxy-5-fluorocytidine (5′-DFCR), doxifluridine (5′-DFUR), capecitabine, and 5-fluorouracil (5-FU). For example, and without limitation, measurements can be made using chromatographic methods (see, e.g., Wang Z et al., J Anal Methods Chem. 2019 Jan. 3; 2019:9371790; Radovanovic M et al., J Chromatogr B Analyt Technol Biomed Life Sci. 2022 January J: 1188:123075; Chen J et al., Bioanalysis. 2010 December; 2 (12): 2011-7; Knikman J E et al., Biomed Chromatogr. 2020 January; 34 (1):e4732; Kaikman, J E et al., Availa able at SSRN ID No. 4193341) and/or Enzyme-linked immunosorbent assay (ELISA) (see, e.g., Blaschke M et al., Journal of Cancer Therapy. Vol. 3 No. 1, 2012, pp. 28-36.).

Methods of the present disclosure can comprise that blood samples obtained from the subject were measured for one or more of: α-fluoro-β-alanine (FBAL), 5′-deoxy-5-fluorocytidine (5′-DFCR), doxifluridine (5′-DFUR), capecitabine, and 5-fluorouracil (5-FU). In some embodiments, a blood sample obtained from the subject was measured for α-fluoro-β-alanine (FBAL) and 5-fluorouracil (5-FU). In some embodiments, a method comprises determining one or more of the measured 5-FU, the measured FBAL, and/or a ratio of measured FBAL to measured 5-FU (FBAL: 5-FU ratio). In some embodiments, a method comprises that a ratio of measured FBAL to measured 5-FU (FBAL: 5-FU ratio) was determined.

In one aspect, the present disclosure provides a method of treating a cancer in a subject in need thereof, comprising: (i) administering at least one dose of an effective amount of PCS6422 to the subject; and (ii) about 12 to about 24 hours after administering the at least one dose of the effective amount of PCS6422, administering at least one dose of a therapeutically effective amount of capecitabine to the subject, wherein a blood sample obtained from the subject was measured for one or more of: α-fluoro-β-alanine (FBAL), 5′-deoxy-5-fluorocytidine (5′-DFCR), doxifluridine (5′-DFUR), capecitabine, and 5-fluorouracil (5-FU).

In another aspect, the present disclosure provides methods of treating cancer in a subject in need thereof comprising: (i) administering at least one dose of an effective amount of PCS6422 to the subject; and (ii) administering at least one dose of a therapeutically effective amount of capecitabine to the subject about 12 to about 36 hours (such as about 12 to about 24 hours) after administering the at least one dose of the effective amount of PCS6422; (iii) measuring metabolites comprising one or more of α-fluoro-β-alanine (FBAL), 5′-deoxy-5-fluorocytidine (5′-DFCR), doxifluridine (5′-DFUR), and 5-fluorouracil (5-FU) in a blood sample obtained from the subject; and (iv) determining whether the therapeutic regimen should be adjusted based on a therapeutic index comprising the metabolite measurements determined in step (iii).

In another aspect, the present disclosure provides methods of determining frequency of PCS6422 and capecitabine administration to a subject comprising: (i) administering at least one dose of an effective amount of PCS6422 to the subject; (ii) about 12 to about 36 hours (such as about 12 to about 24 hours) after administering the at least one dose of the effective amount of PCS6422, administering at least one dose of a therapeutically effective amount of capecitabine to the subject, wherein a blood sample obtained from the subject was measured for α-fluoro-β-alanine (FBAL) and 5-fluorouracil (5-FU); (iii) determining one or more of the measured 5-FU, the measured FBAL, and/or a ratio of measured FBAL to measured 5-FU (FBAL: 5-FU ratio); and (iv) after a drug-free period, repeating steps (i) and (ii) when at least one of the following conditions are met: the measured 5-FU was less than a certain 5-FU threshold, the measured FBAL is greater than or equal to a certain FBAL threshold, the FBAL: 5-FU ratio determined was greater than or equal to a certain FBAL: 5-FU ratio threshold, the measured 5-FU is zero, or the measured FBAL is zero.

In yet another aspect, the present disclosure provides methods of determining frequency of capecitabine administration to a subject comprising (i) administering at least one dose of an effective amount of PCS6422 to the subject; (ii) about 12 to about 36 hours (such as about 12 to about 24 hours) after administering the at least one dose of the effective amount of PCS6422, administering at least one dose of a therapeutically effective amount of capecitabine to the subject, wherein a blood sample obtained from the subject was measured for α-fluoro-β-alanine (FBAL) and/or 5-fluorouracil (5-FU); (iii) determining one or more of the measured 5-FU, the measured FBAL, or a ratio of measured FBAL to measured 5-FU (FBAL: 5-FU ratio); and (iv) stopping administering an additional dose of the therapeutically effective amount of capecitabine to the subject when the measured 5-FU was less than a certain 5-FU threshold, the measured FBAL is greater than or equal to a certain FBAL threshold, or the FBAL: 5-FU ratio determined was greater than or equal to a certain FBAL: 5-FU ratio threshold.

In some embodiments, methods of the disclosure can comprise stopping administering an additional dose of the therapeutically effective amount of capecitabine to the subject when the measured 5-FU was less than a certain 5-FU threshold, the measured FBAL is greater than or equal to a certain FBAL threshold, or the FBAL: 5-FU ratio determined was greater than or equal to a certain FBAL: 5-FU ratio threshold. In some such embodiments, stopping administering an additional dose of the therapeutically effective amount of capecitabine to the subject is followed by a drug-free period (e.g., as described herein). In some embodiments, after the drug-free period, steps (i) and (ii) are repeated (e.g., when the measured 5-FU is zero and/or the measured FBAL is zero).

In some embodiments, about 1 day to about 29 days after step (ii) of methods described herein, steps (i) and (ii) can be repeated when at least one of the following conditions are met: the measured 5-FU was less than a certain 5-FU threshold, the measured FBAL is greater than or equal to a certain FBAL threshold, the FBAL: 5-FU ratio determined was greater than or equal to a certain FBAL: 5-FU ratio threshold, the measured 5-FU is zero, or the measured FBAL is zero.

In some embodiments, about 1 day to about 29 days after step (ii) of methods described herein, steps (i) and (ii) can be repeated when the therapeutic index including the metabolite measurement in (iii) indicates that, among other therapeutic regimen-determining factors described herein, the measured 5-FU was less than a certain 5-FU threshold, the measured FBAL is greater than or equal to a certain FBAL threshold, the FBAL: 5-FU ratio determined was greater than or equal to a certain FBAL: 5-FU ratio threshold, the measured 5-FU is zero, or the measured FBAL is zero.

In some embodiments, about 1 day to about 29 days after step (ii) of methods described herein, steps (i) and (ii) can be repeated when the FBAL: 5-FU ratio determined was greater than or equal to a certain FBAL: 5-FU ratio threshold. In some embodiments, about 1 day to about 22 days after step (ii), steps (i) and (ii) are repeated when the FBAL: 5-FU ratio determined was greater than or equal to a certain FBAL: 5-FU ratio threshold. In some embodiments, about 1 day to about 15 days after step (ii), steps (i) and (ii) are repeated when the FBAL: 5-FU ratio determined was greater than or equal to a certain FBAL: 5-FU ratio threshold. In some embodiments, about 1 day to about 12 days after step (ii), steps (i) and (ii) are repeated when the FBAL: 5-FU ratio determined was greater than or equal to a certain FBAL: 5-FU ratio threshold. In some embodiments, about 1 day to about 10 days after step (ii), steps (i) and (ii) are repeated when the FBAL: 5-FU ratio determined was greater than or equal to a certain FBAL: 5-FU ratio threshold. In some embodiments, about 1 day to about 8 days after step (ii), steps (i) and (ii) are repeated when the FBAL: 5-FU ratio determined was greater than or equal to a certain FBAL: 5-FU ratio threshold. In some embodiments, about 1 day to about 6 days after step (ii), steps (i) and (ii) are repeated when the FBAL: 5-FU ratio determined was greater than or equal to a certain FBAL: 5-FU ratio threshold. In some embodiments, about 1 day to about 4 days after step (ii), steps (i) and (ii) are repeated when the FBAL: 5-FU ratio determined was greater than or equal to a certain FBAL: 5-FU ratio threshold. In some embodiments, about 1 day to about 2 days after step (ii), steps (i) and (ii) are repeated when the FBAL: 5-FU ratio determined was greater than or equal to a certain FBAL: 5-FU ratio threshold. In some embodiments, about 1 day after step (ii), steps (i) and (ii) are repeated when the FBAL: 5-FU ratio determined was greater than or equal to a certain FBAL: 5-FU ratio threshold. In some embodiments, about 2 days after step (ii), steps (i) and (ii) are repeated when the FBAL: 5-FU ratio determined was greater than or equal to a certain FBAL: 5-FU ratio threshold. In some embodiments, about 3 days after step (ii), steps (i) and (ii) are repeated when the FBAL: 5-FU ratio determined was greater than or equal to a certain FBAL: 5-FU ratio threshold. In some embodiments, about 4 days after step (ii), steps (i) and (ii) are repeated when the FBAL: 5-FU ratio determined was greater than or equal to a certain FBAL: 5-FU ratio threshold. In some embodiments, about 5 days after step (ii), steps (i) and (ii) are repeated when the FBAL: 5-FU ratio determined was greater than or equal to a certain FBAL: 5-FU ratio threshold. In some embodiments, about 6 days after step (ii), steps (i) and (ii) are repeated when the FBAL: 5-FU ratio determined was greater than or equal to a certain FBAL: 5-FU ratio threshold. In some embodiments, about 7 days after step (ii), steps (i) and (ii) are repeated when the FBAL: 5-FU ratio determined was greater than or equal to a certain FBAL: 5-FU ratio threshold. In some embodiments, about 8 days after step (ii), steps (i) and (ii) are repeated when the FBAL: 5-FU ratio determined was greater than or equal to a certain FBAL: 5-FU ratio threshold. In some embodiments, about 10 days after step (ii), steps (i) and (ii) are repeated when the FBAL: 5-FU ratio determined was greater than or equal to a certain FBAL: 5-FU ratio threshold. In some embodiments, about 12 days after step (ii), steps (i) and (ii) are repeated when the FBAL: 5-FU ratio determined was greater than or equal to a certain FBAL: 5-FU ratio threshold. In some embodiments, about 14 days after step (ii), steps (i) and (ii) are repeated when the FBAL: 5-FU ratio determined was greater than or equal to a certain FBAL: 5-FU ratio threshold. In some embodiments, about 15 days after step (ii), steps (i) and (ii) are repeated when the FBAL: 5-FU ratio determined was greater than or equal to a certain FBAL: 5-FU ratio threshold. In some embodiments, about 21 days after step (ii), steps (i) and (ii) are repeated when the FBAL: 5-FU ratio determined was greater than or equal to a certain FBAL: 5-FU ratio threshold. In some embodiments, about 22 days after step (ii), steps (i) and (ii) are repeated when the FBAL: 5-FU ratio determined was greater than or equal to a certain FBAL: 5-FU ratio threshold. In some embodiments, about 28 days after step (ii), steps (i) and (ii) are repeated when the FBAL: 5-FU ratio determined was greater than or equal to a certain FBAL: 5-FU ratio threshold. In some embodiments, about 29 days after step (ii), steps (i) and (ii) are repeated when the FBAL: 5-FU ratio determined was greater than or equal to a certain FBAL: 5-FU ratio threshold.

In some embodiments, steps (i) and (ii) are not repeated when the FBAL: 5-FU ratio determined was less than a certain FBAL: 5-FU ratio threshold.

In some embodiments, after step (ii) of methods described herein, administering an additional dose of the therapeutically effective amount of capecitabine to the subject is stopped when the FBAL: 5-FU ratio determined was greater than or equal to a certain FBAL: 5-FU ratio threshold.

In some embodiments, after step (ii) of methods described herein, administering an additional dose of the therapeutically effective amount of capecitabine to the subject is continued when the FBAL: 5-FU ratio determined was less than a certain FBAL: 5-FU ratio threshold.

The certain FBAL: 5-FU ratio threshold can be determined as about 2 to about 15, about 4 to about 15, about 6 to about 15, about 2 to about 12, about 4 to about 12, about 6 to about 12, about 2 to about 10, about 4 to about 10, or about 6 to about 10. In some embodiments, the certain FBAL: 5-FU ratio threshold can be determined as about 5, about 5.5, about 6, about 6.5, about 7, about 7.5, about 8, about 8.5, about 9, about 9.5, about 10, about 10.5, about 11, about 11.5, about 12, about 12.5, about 13, about 13.5, about 14, about 14.5 or about 15. In some embodiments, the certain FBAL: 5-FU ratio threshold can be determined as about 5. In some embodiments, the certain FBAL: 5-FU ratio threshold can be determined as about 5.5. In some embodiments, the certain FBAL: 5-FU ratio threshold can be determined as about 6. In some embodiments, the certain FBAL: 5-FU ratio threshold can be determined as about 6.5. In some embodiments, the certain FBAL: 5-FU ratio threshold can be determined as about 7. In some embodiments, the certain FBAL: 5-FU ratio threshold can be determined as about 7.5. In some embodiments, the certain FBAL: 5-FU ratio threshold can be determined as about 8. In some embodiments, the certain FBAL: 5-FU ratio threshold can be determined as about 8.5. In some embodiments, the certain FBAL: 5-FU ratio threshold can be determined as about 9. In some embodiments, the certain FBAL: 5-FU ratio threshold can be determined as about 9.5. In some embodiments, the certain FBAL: 5-FU ratio threshold can be determined as about 10.

In some embodiments, about 1 day to about 29 days after step (ii) of methods described herein, steps (i) and (ii) can be repeated when the measured 5-FU determined was less than a certain 5-FU threshold. In some embodiments, about 1 day to about 22 days after step (ii) of methods described herein, steps (i) and (ii) can be repeated when the measured 5-FU determined was less than a certain 5-FU threshold. In some embodiments, about 1 day to about 15 days after step (ii) of methods described herein, steps (i) and (ii) can be repeated when the measured 5-FU determined was less than a certain 5-FU threshold. In some embodiments, about 1 day to about 12 days after step (ii), steps (i) and (ii) are repeated when the measured 5-FU determined was less than a certain 5-FU threshold. In some embodiments, about 1 day to about 10 days after step (ii), steps (i) and (ii) are repeated when the measured 5-FU determined was less than a certain 5-FU threshold. In some embodiments, about 1 day to about 8 days after step (ii), steps (i) and (ii) are repeated when the measured 5-FU determined was less than a certain 5-FU threshold. In some embodiments, about 1 day to about 6 days after step (ii), steps (i) and (ii) are repeated when the measured 5-FU determined was less than a certain 5-FU threshold. In some embodiments, about 1 day to about 4 days after step (ii), steps (i) and (ii) are repeated when the measured 5-FU determined was less than a certain 5-FU threshold. In some embodiments, about 1 day to about 2 days after step (ii), steps (i) and (ii) are repeated when the measured 5-FU determined was less than a certain 5-FU threshold. In some embodiments, about 1 day after step (ii), steps (i) and (ii) are repeated when the measured 5-FU determined was less than a certain 5-FU threshold. In some embodiments, about 2 days after step (ii), steps (i) and (ii) are repeated when the measured 5-FU determined was less than a certain 5-FU threshold. In some embodiments, about 3 days after step (ii), steps (i) and (ii) are repeated when the measured 5-FU determined was less than a certain 5-FU threshold. In some embodiments, about 4 days after step (ii), steps (i) and (ii) are repeated when the measured 5-FU determined was less than a certain 5-FU threshold. In some embodiments, about 5 days after step (ii), steps (i) and (ii) are repeated when the measured 5-FU determined was less than a certain 5-FU threshold. In some embodiments, about 6 days after step (ii), steps (i) and (ii) are repeated when the measured 5-FU determined was less than a certain 5-FU threshold. In some embodiments, about 7 days after step (ii), steps (i) and (ii) are repeated when the measured 5-FU determined was less than a certain 5-FU threshold. In some embodiments, about 8 days after step (ii), steps (i) and (ii) are repeated when the measured 5-FU determined was less than a certain 5-FU threshold. In some embodiments, about 10 days after step (ii), steps (i) and (ii) are repeated when the measured 5-FU determined was less than a certain 5-FU threshold. In some embodiments, about 12 days after step (ii), steps (i) and (ii) are repeated when the measured 5-FU determined was less than a certain 5-FU threshold. In some embodiments, about 14 days after step (ii), steps (i) and (ii) are repeated when the measured 5-FU determined was less than a certain 5-FU threshold. In some embodiments, about 15 days after step (ii), steps (i) and (ii) are repeated when the measured 5-FU determined was less than a certain 5-FU threshold. In some embodiments, about 21 days after step (ii), steps (i) and (ii) are repeated when the measured 5-FU determined was less than a certain 5-FU threshold. In some embodiments, about 22 days after step (ii), steps (i) and (ii) are repeated when the measured 5-FU determined was less than a certain 5-FU threshold. In some embodiments, about 28 days after step (ii), steps (i) and (ii) are repeated when the measured 5-FU determined was less than a certain 5-FU threshold. In some embodiments, about 29 days after step (ii), steps (i) and (ii) are repeated when the measured 5-FU determined was less than a certain 5-FU threshold.

In some embodiments, steps (i) and (ii) are not repeated when the measured 5-FU determined was greater than or equal to a certain 5-FU threshold.

In some embodiments, about 1 day to about 29 days after step (ii) of methods described herein, steps (i) and (ii) can be repeated when the measured 5-FU determined was zero. In some embodiments, about 1 day to about 22 days after step (ii), steps (i) and (ii) are repeated when the measured 5-FU determined was zero. In some embodiments, about 1 day to about 15 days after step (ii), steps (i) and (ii) are repeated when the measured 5-FU determined was zero. In some embodiments, about 1 day to about 12 days after step (ii), steps (i) and (ii) are repeated when the measured 5-FU determined was zero. In some embodiments, about 1 day to about 10 days after step (ii), steps (i) and (ii) are repeated when the measured 5-FU determined was zero. In some embodiments, about 1 day to about 8 days after step (ii), steps (i) and (ii) are repeated when the measured 5-FU determined was zero. In some embodiments, about 1 day to about 6 days after step (ii), steps (i) and (ii) are repeated when the measured 5-FU determined was zero. In some embodiments, about 1 day to about 4 days after step (ii), steps (i) and (ii) are repeated when the measured 5-FU determined was zero. In some embodiments, about 1 day to about 2 days after step (ii), steps (i) and (ii) are repeated when the measured 5-FU determined was zero. In some embodiments, about 1 day after step (ii), steps (i) and (ii) are repeated when the measured 5-FU determined was zero. In some embodiments, about 2 days after step (ii), steps (i) and (ii) are repeated when the measured 5-FU determined was zero. In some embodiments, about 3 days after step (ii), steps (i) and (ii) are repeated when the measured 5-FU determined was zero. In some embodiments, about 4 days after step (ii), steps (i) and (ii) are repeated when the measured 5-FU determined was zero. In some embodiments, about 5 days after step (ii), steps (i) and (ii) are repeated when the measured 5-FU determined was zero. In some embodiments, about 6 days after step (ii), steps (i) and (ii) are repeated when the measured 5-FU determined was zero. In some embodiments, about 7 days after step (ii), steps (i) and (ii) are repeated when the measured 5-FU determined was zero. In some embodiments, about 8 days after step (ii), steps (i) and (ii) are repeated when the measured 5-FU determined was zero. In some embodiments, about 10 days after step (ii), steps (i) and (ii) are repeated when the measured 5-FU determined was zero. In some embodiments, about 12 days after step (ii), steps (i) and (ii) are repeated when the measured 5-FU determined was zero. In some embodiments, about 14 days after step (ii), steps (i) and (ii) are repeated when the measured 5-FU determined was zero. In some embodiments, about 15 days after step (ii), steps (i) and (ii) are repeated when the measured 5-FU determined was zero. In some embodiments, about 21 days after step (ii), steps (i) and (ii) are repeated when the measured 5-FU determined was zero. In some embodiments, about 22 days after step (ii), steps (i) and (ii) are repeated when the measured 5-FU determined was zero. In some embodiments, about 28 days after step (ii), steps (i) and (ii) are repeated when the measured 5-FU determined was zero. In some embodiments, about 29 days after step (ii), steps (i) and (ii) are repeated when the measured 5-FU determined was zero.

In some embodiments, steps (i) and (ii) are not repeated when the measured 5-FU determined was greater than zero.

In some embodiments, after step (ii) of methods described herein, administering an additional dose of the therapeutically effective amount of capecitabine to the subject is stopped when the measured 5-FU determined was less than a certain 5-FU threshold.

In some embodiments, after step (ii) of methods described herein, administering an additional dose of the therapeutically effective amount of capecitabine to the subject is continued when the measured 5-FU determined was greater than or equal to a certain 5-FU threshold.

The certain 5-FU threshold can be determined as about 0 ng/ml to about 5 ng/ml, about 0 ng/ml to about 4 ng/ml, about 0 ng/mL to about 3 ng/ml, about 1 ng/ml to about 5 ng/mL, about 1 to about 4 ng/ml, about 1 ng/mL to about 3 ng/mL, about 1.5 ng/ml to about 5 ng/ml, about 1.5 ng/ml to about 4 ng/ml, or about 1.5 ng/ml to about 3 ng/ml. In some embodiments, the certain 5-FU threshold can be determined as about 0.5 ng/ml, about 1 ng/mL, about 1.5 ng/mL. about 2 ng/ml, about 2.5 ng/mL, about 3 ng/ml, about 3.5 ng/ml, about 4 ng/ml, about 4.5 ng/ml, or about 5 ng/mL. In some embodiments, the certain 5-FU threshold can be determined as about 0 ng/mL. In some embodiments, the certain 5-FU threshold can be determined as about 0.5 ng/mL. In some embodiments, the certain 5-FU threshold can be determined as about 1 ng/mL. In some embodiments, the certain 5-FU threshold can be determined as about 2 ng/mL. In some embodiments, the certain 5-FU threshold can be determined as about 3 ng/mL. In some embodiments, the certain 5-FU threshold can be determined as about 4 ng/mL. In some embodiments, the certain 5-FU threshold can be determined as about 5 ng/ml.

In some embodiments, about 1 day to about 29 days after step (ii) of methods described herein, steps (i) and (ii) can be repeated when the measured FBAL determined was greater than or equal to a certain FBAL threshold. In some embodiments, about 1 day to about 22 days after step (ii) of methods described herein, steps (i) and (ii) can be repeated when the measured FBAL determined was greater than or equal to a certain FBAL threshold. In some embodiments, about 1 day to about 15 days after step (ii) of methods described herein, steps (i) and (ii) can be repeated when the measured FBAL determined was greater than or equal to a certain FBAL threshold. In some embodiments, about 1 day to about 12 days after step (ii), steps (i) and (ii) are repeated when the measured FBAL determined was greater than or equal to a certain FBAL threshold. In some embodiments, about 1 day to about 10 days after step (ii), steps (i) and (ii) are repeated when the measured FBAL determined was greater than or equal to a certain FBAL threshold. In some embodiments, about 1 day to about 8 days after step (ii), steps (i) and (ii) are repeated when the measured FBAL determined was greater than or equal to a certain FBAL threshold. In some embodiments, about 1 day to about 6 days after step (ii), steps (i) and (ii) are repeated when the measured FBAL determined was greater than or equal to a certain FBAL threshold. In some embodiments, about 1 day to about 4 days after step (ii), steps (i) and (ii) are repeated when the measured FBAL determined was greater than or equal to a certain FBAL threshold. In some embodiments, about 1 day to about 2 days after step (ii), steps (i) and (ii) are repeated when the measured FBAL determined was greater than or equal to a certain FBAL threshold. In some embodiments, about 1 day after step (ii), steps (i) and (ii) are repeated when the measured FBAL determined was greater than or equal to a certain FBAL threshold. In some embodiments, about 2 days after step (ii), steps (i) and (ii) are repeated when the measured FBAL determined was greater than or equal to a certain FBAL threshold. In some embodiments, about 3 days after step (ii), steps (i) and (ii) are repeated when the measured FBAL determined was greater than or equal to a certain FBAL threshold. In some embodiments, about 4 days after step (ii), steps (i) and (ii) are repeated when the measured FBAL determined was greater than or equal to a certain FBAL threshold. In some embodiments, about 5 days after step (ii), steps (i) and (ii) are repeated when the measured FBAL determined was greater than or equal to a certain FBAL threshold. In some embodiments, about 6 days after step (ii), steps (i) and (ii) are repeated when the measured FBAL determined was greater than or equal to a certain FBAL threshold. In some embodiments, about 7 days after step (ii), steps (i) and (ii) are repeated when the measured FBAL determined was greater than or equal to a certain FBAL threshold. In some embodiments, about 8 days after step (ii), steps (i) and (ii) are repeated when the measured FBAL determined was greater than or equal to a certain FBAL threshold. In some embodiments, about 10 days after step (ii), steps (i) and (ii) are repeated when the measured FBAL determined was greater than or equal to a certain FBAL threshold. In some embodiments, about 12 days after step (ii), steps (i) and (ii) are repeated when the measured 5-FBAL determined was greater than or equal to a certain FBAL threshold. In some embodiments, about 14 days after step (ii), steps (i) and (ii) are repeated when the measured FBAL determined was greater than or equal to a certain FBAL threshold. In some embodiments, about 15 days after step (ii), steps (i) and (ii) are repeated when the measured FBAL determined was greater than or equal to a certain FBAL threshold. In some embodiments, about 21 days after step (ii), steps (i) and (ii) are repeated when the measured FBAL determined was greater than or equal to a certain FBAL threshold. In some embodiments, about 22 days after step (ii), steps (i) and (ii) are repeated when the measured FBAL determined was greater than or equal to a certain FBAL threshold. In some embodiments, about 28 days after step (ii), steps (i) and (ii) are repeated when the measured FBAL determined was greater than or equal to a certain FBAL threshold. In some embodiments, about 29 days after step (ii), steps (i) and (ii) are repeated when the measured FBAL determined was greater than or equal to a certain FBAL threshold.

In some embodiments, steps (i) and (ii) are not repeated when the measured FBAL determined was less than a certain FBAL threshold.

In some embodiments, about 1 day to about 29 days after step (ii) of methods described herein, steps (i) and (ii) can be repeated when the measured FBAL determined was zero. In some embodiments, about 1 day to about 22 days after step (ii) of methods described herein, steps (i) and (ii) can be repeated when the measured FBAL determined was zero. In some embodiments, about 1 day to about 15 days after step (ii) of methods described herein, steps (i) and (ii) can be repeated when the measured FBAL determined was zero. In some embodiments, about 1 day to about 12 days after step (ii), steps (i) and (ii) are repeated when the measured FBAL determined was zero. In some embodiments, about 1 day to about 10 days after step (ii), steps (i) and (ii) are repeated when the measured FBAL determined was zero. In some embodiments, about 1 day to about 8 days after step (ii), steps (i) and (ii) are repeated when the measured FBAL determined was zero. In some embodiments, about 1 day to about 6 days after step (ii), steps (i) and (ii) are repeated when the measured FBAL determined was zero. In some embodiments, about 1 day to about 4 days after step (ii), steps (i) and (ii) are repeated when the measured FBAL determined was zero. In some embodiments, about 1 day to about 2 days after step (ii), steps (i) and (ii) are repeated when the measured FBAL determined was zero. In some embodiments, about 1 day after step (ii), steps (i) and (ii) are repeated when the measured FBAL determined was zero. In some embodiments, about 2 days after step (ii), steps (i) and (ii) are repeated when the measured FBAL determined was zero. In some embodiments, about 3 days after step (ii), steps (i) and (ii) are repeated when the measured FBAL determined was zero. In some embodiments, about 4 days after step (ii), steps (i) and (ii) are repeated when the measured FBAL determined was zero. In some embodiments, about 5 days after step (ii), steps (i) and (ii) are repeated when the measured FBAL determined was zero. In some embodiments, about 6 days after step (ii), steps (i) and (ii) are repeated when the measured FBAL determined was zero. In some embodiments, about 7 days after step (ii), steps (i) and (ii) are repeated when the measured FBAL determined was zero. In some embodiments, about 8 days after step (ii), steps (i) and (ii) are repeated when the measured FBAL determined was zero. In some embodiments, about 10 days after step (ii), steps (i) and (ii) are repeated when the measured FBAL determined was zero. In some embodiments, about 12 days after step (ii), steps (i) and (ii) are repeated when the measured 5-FBAL determined was zero. In some embodiments, about 14 days after step (ii), steps (i) and (ii) are repeated when the measured FBAL determined was zero. In some embodiments, about 15 days after step (ii), steps (i) and (ii) are repeated when the measured FBAL determined was zero. In some embodiments, about 21 days after step (ii), steps (i) and (ii) are repeated when the measured FBAL determined was zero. In some embodiments, about 22 days after step (ii), steps (i) and (ii) are repeated when the measured FBAL determined was zero. In some embodiments, about 28 days after step (ii), steps (i) and (ii) are repeated when the measured FBAL determined was zero. In some embodiments, about 29 days after step (ii), steps (i) and (ii) are repeated when the measured FBAL determined was zero.

In some embodiments, steps (i) and (ii) are not repeated when the measured FBAL determined was greater than zero.

In some embodiments, after step (ii) of methods described herein, administering an additional dose of the therapeutically effective amount of capecitabine to the subject is stopped when the measured FBAL determined was greater than or equal to a certain FBAL threshold.

In some embodiments, after step (ii) of methods described herein, administering an additional dose of the therapeutically effective amount of capecitabine to the subject is continued when the measured FBAL determined was less than a certain FBAL threshold.

The certain FBAL threshold can be determined as about 5 ng/mL to about 50 ng/ml, about 10 ng/ml to about 50 ng/ml, about 15 ng/ml to about 50 ng/ml, about 5 ng/ml to about 40 ng/mL, about 10 ng/ml to about 40 ng/mL, or about 15 ng/ml to about 40 ng/ml, about 5 ng/mL to about 30 ng/mL, about 10 ng/ml to about 30 ng/ml, or about 15 ng/ml to about 30 ng/mL. In some embodiments, the certain FBAL threshold can be determined as about 5 ng/ml, about 10 ng/ml, about 15 ng/mL. about 20 ng/ml, about 25 ng/ml, about 30 ng/ml, about 35 ng/mL, about 40 ng/mL, about 45 ng/ml, or about 50 ng/mL. In some embodiments, the certain FBAL threshold can be determined as about 5 ng/mL. In some embodiments, the certain FBAL threshold can be determined as about 10 ng/mL. In some embodiments, the certain FBAL threshold can be determined as about 15 ng/mL. In some embodiments, the certain FBAL threshold can be determined as about 20 ng/mL. In some embodiments, the certain FBAL threshold can be determined as about 25 ng/ml. In some embodiments, the certain FBAL threshold can be determined as about 30 ng/mL. In some embodiments, the certain FBAL threshold can be determined as about 35 ng/mL. In some embodiments, the certain FBAL threshold can be determined as about 40 ng/mL. In some embodiments, the certain FBAL threshold can be determined as about 45 ng/mL. In some embodiments, the certain FBAL threshold can be determined as about 50 ng/mL. In some embodiments, the certain FBAL threshold can be determined as about 0 ng/mL

A blood sample obtained from the subject for use in accordance with methods of the present disclosure can be obtained at a single time point or a plurality of time points over the course of a treatment cycle. In some embodiments, a blood sample obtained from the subject can be obtained in the morning (e.g., before administration of a dose of a therapeutically effective amount of capecitabine to the subject). In some embodiments, a blood sample obtained from the subject can be obtained in the evening (e.g., before administration of a divided dose of a therapeutically effective amount of capecitabine to the subject and/or approximately 12 hours after a morning dose of a therapeutically effective amount of capecitabine). In some embodiments, a blood sample obtained from the subject can be obtained about 24 hours after administration of an effective amount of PCS6422.

A blood sample obtained from the subject can be obtained about 12 to about 24 hours after administering a therapeutically effective amount of capecitabine to the subject (e.g., but before any additional doses of capecitabine are administered, e.g., at the Cₘᵢₙ). In some embodiments, a blood sample obtained from the subject was obtained daily after the administration of at least one dose of the therapeutically effective amount of capecitabine. In some embodiments, a blood sample obtained from the subject was obtained on each of Days 2-29 of a treatment cycle. In some embodiments, a blood sample obtained from the subject was obtained on each of Days 2-28 of a treatment cycle. In some embodiments, a blood sample obtained from the subject was obtained on each of Days 2-22 of a treatment cycle. In some embodiments, a blood sample obtained from the subject was obtained on each of Days 2-21 of a treatment cycle. In some embodiments, a blood sample obtained from the subject was obtained on each of Days 2-15 of a treatment cycle. In some embodiments, a blood sample obtained from the subject was obtained on each of Days 2-14 of a treatment cycle. In some embodiments, a blood sample obtained from the subject was obtained on each of Days 2-10 of a treatment cycle. In some embodiments, a blood sample obtained from the subject was obtained on each of Days 2-9 of a treatment cycle. In some embodiments, a blood sample obtained from the subject was obtained on each of Days 2-8 of a treatment cycle. In some embodiments, a blood sample obtained from the subject was obtained on each of Days 2-7 of a treatment cycle. In some embodiments, a blood sample obtained from the subject was obtained on each of Days 2-6 of a treatment cycle. In some embodiments, a blood sample obtained from the subject was obtained on each of Days 2-5 of a treatment cycle. In some embodiments, a blood sample obtained from the subject was obtained on each of Days 2-4 of a treatment cycle. In some embodiments, a blood sample obtained from the subject was obtained on each of Days 2-3 of a treatment cycle. In some embodiments, a blood sample obtained from the subject was obtained on each of Days 3-10 of a treatment cycle. In some embodiments, a blood sample obtained from the subject was obtained on each of Days 3-9 of a treatment cycle. In some embodiments, a blood sample obtained from the subject was obtained on each of Days 3-8 of a treatment cycle. In some embodiments, a blood sample obtained from the subject was obtained on each of Days 3-7 of a treatment cycle. In some embodiments, a blood sample obtained from the subject was obtained on each of Days 3-6 of a treatment cycle. In some embodiments, a blood sample obtained from the subject was obtained on each of Days 3-5 of a treatment cycle. In some embodiments, a blood sample obtained from the subject was obtained on each of Days 3-4 of a treatment cycle. In some embodiments, a blood sample obtained from the subject was obtained on each of Days 4-10 of a treatment cycle. In some embodiments, a blood sample obtained from the subject was obtained on each of Days 4-9 of a treatment cycle. In some embodiments, a blood sample obtained from the subject was obtained on each of Days 4-8 of a treatment cycle. In some embodiments, a blood sample obtained from the subject was obtained on each of Days 4-7 of a treatment cycle. In some embodiments, a blood sample obtained from the subject was obtained on each of Days 4-6 of a treatment cycle. In some embodiments, a blood sample obtained from the subject was obtained on each of Days 4-5 of a treatment cycle. In some embodiments, a blood sample obtained from the subject was obtained on each of Days 2-4 and 6-14 of a treatment cycle. In some embodiments, a blood sample obtained from the subject was obtained on each of Days 2-7, 9-14, and 16-28 of a treatment cycle.

In some embodiments, one or more of FBAL, 5′-DFCR, 5′-DFUR, capecitabine, and 5-FU were measured daily starting on the first day of capecitabine treatment (e.g., prior to the initial dose of capecitabine administered). In some embodiments, FBAL and 5-FU were measured daily starting on the first day of capecitabine treatment (e.g., prior to the initial dose of capecitabine administered). In some embodiments, one or more of FBAL, 5′-DFCR, 5′-DFUR, capecitabine, and 5-FU were measured at each time point in which a blood sample was obtained from the subject.

### Subject Population

A subject and/or a subject in need thereof in accordance with the technologies described herein include, but are not limited to, humans and non-human vertebrates. In some embodiments, a subject and/or a subject in need thereof in accordance with technologies disclosed herein include, for example, a mammal. Mammals include, for example and without limitation, a household pet (e.g., a dog, a cat, a rabbit, a hamster), a livestock and/or farm animal (e.g., a cow, a pig, a sheep, a goat, a chicken or another poultry), a horse, a monkey, a laboratory animal (e.g., a mouse, a rat, a rabbit), and the like. In a preferred embodiment, the subject and/or subject in need thereof in accordance with the methods described herein is a human.

A subject and/or subject in need thereof of the present disclosure can be an adult (e.g., a human adult). In some embodiments, the subject and/or subject in need thereof is a human subject over 18 years of age. In some embodiments, the subject and/or subject in need thereof is a human subject over 21 years of age. In some embodiments, the subject and/or subject in need thereof is a human subject over 45 years of age. In some embodiments, the subject and/or subject in need thereof is a human subject over 65 years of age. In some embodiments, the subject and/or subject in need thereof is a human subject under 45 years of age (or between 18 and 45 years of age, or between 21 and 45 years of age). In some embodiments, the subject and/or subject in need thereof is a human subject under 65 years of age (or between 18 and 65 years of age, between 21 and 65 years of age, or between 45 and 65 years of age).

A subject and/or a subject in need thereof of the present disclosure can be a non-adult (e.g., a pediatric human, an adolescent human). In some embodiments, the subject and/or subject in need thereof is a human subject under 18 years of age. In some embodiments, the subject and/or subject in need thereof is a human subject under 12 years of age. In some embodiments, the subject and/or subject in need thereof is a human subject under 5 years of age.

In some aspects, the methods described herein can be utilized and/or practiced in any subject that has (e.g., has been diagnosed with) a cancer (e.g., a cancer that may and/or is likely to benefit from the methods described herein). A subject having a tumor (e.g., a solid tumor) is a subject that has detectable tumor cells. The present disclosure contemplates, among other things, use of any method described herein to treat a cancer in a subject in need thereof and/or to determine frequency of (i) PCS6422 and capecitabine or (ii) capecitabine administration to a subject (e.g., a subject in need thereof).

In some aspects, the methods of the present disclosure can be utilized in a subject and/or a subject in need thereof that has a cancer. A subject and/or a subject in need thereof that has cancer has detectable cancer cells. Examples of cancers that may, in some embodiments, be treated in accordance with technologies of the present disclosure include, without limitation, adrenocortical carcinoma, astrocytoma, basal cell carcinoma, breast cancers, carcinoid, cardiac, cholangiocarcinoma, chordoma, chronic myeloproliferative neoplasms, craniopharyngioma, ductal carcinoma in situ, ependymoma, intraocular melanoma, gastrointestinal tract tumors (e.g., gastrointestinal carcinoid tumor, colorectal tumor, gastrointestinal stromal tumor (GIST)), gestational trophoblastic disease, glioma, histiocytosis, leukemia (e.g., acute lymphoblastic leukemia (ALL), acute myeloid leukemia (AML), chronic lymphocytic leukemia (CLL), chronic myelogenous leukemia (CML), hairy cell leukemia, myelogenous leukemia, and myeloid leukemia), lymphoma (e.g., Burkitt lymphoma (non-Hodgkin lymphoma), cutaneous T-cell lymphoma, Hodgkin lymphoma, mycosis fungoides, Sezary syndrome, AIDS-related lymphoma, follicular lymphoma, diffuse large B-cell lymphoma), melanoma, merkel cell carcinoma, mesothelioma, myeloma (e.g., multiple myeloma), myelodysplastic syndrome, pancreatic tumors, papillomatosis, paraganglioma, pheochromocytoma, pleuropulmonary blastoma, retinoblastoma, sarcoma (e.g., Ewing sarcoma, Kaposi sarcoma, osteosarcoma, rhabdomyosarcoma, uterine sarcoma, vascular sarcoma), Wilms' tumor, and/or cancer of the adrenal cortex, anus, appendix, bile duct, bladder, bone, brain, breast, bronchus, central nervous system, cervix, colon, colorectum, endometrium, esophagus, eye, fallopian tube, gall bladder, gastrointestinal tract, germ cell, head and neck, heart, intestine, kidney (e.g., Wilms' tumor), larynx, liver, lung (e.g., non-small cell lung cancer, small cell lung cancer), mouth, nasal cavity, oral cavity, ovary, pancreas, rectum, skin, stomach, testes, throat, thyroid, penis, pharynx, peritoneum, pituitary, prostate, rectum, salivary gland, ureter, urethra, uterus, vagina, or vulva. In some embodiments, a subject and/or a subject in need thereof has a colorectal cancer (e.g., metastatic colorectal cancer). In some embodiments, a subject and/or a subject in need thereof has a gastrointestinal cancer (e.g., metastatic gastrointestinal cancer). In some embodiments, a subject and/or a subject in need thereof has a breast cancer (e.g., metastatic breast cancer). In some embodiments, a subject and/or a subject in need thereof has a pancreatic cancer (e.g., metastatic pancreatic cancer).

Cancers of the present disclosure may involve one or more tumors, such as a solid tumor. Solid tumors can include, but are not limited to, tumors of the bladder, breast, central nervous system, cervix, colon, colorectum, esophagus, endometrium, gastrointestinal tract, head and neck, liver, lung, ovary, pancreas, skin, stomach, uterus, and/or upper respiratory tract.

Cancers of the present disclosure may be advanced in stage (e.g., metastatic, stage 3 cancer, stage 4 cancer). Advanced cancers may be considered and/or determined (e.g., by an ordinary medical practitioners) to be unlikely to be cured and/or controlled with therapeutic intervention. Cancers of the present disclosure may be refractory (e.g., a cancer that has been unresponsive to treatment).

Tests for diagnosing the cancers to be treated by the methods described herein are known in the art and will be familiar to the ordinary medical practitioner. These laboratory tests include, without limitation, microscopic analyses, cultivation dependent tests (such as cultures), and nucleic acid detection tests. These include wet mounts, stain-enhanced microscopy, immune microscopy (e.g., FISH), hybridization microscopy, particle agglutination, enzyme-linked immunosorbent assays, urine screening tests, DNA probe hybridization, serologic tests, etc. The medical practitioner generally takes a full history and conducts a complete physical examination in addition to running the laboratory tests listed above.

In some aspects, the subject and/or subject in need thereof has previously undergone one or more other cancer therapies (e.g., chemotherapy, radiotherapy, or surgical tumor resection). In certain embodiments, the subject and/or subject in need thereof has previously undergone one or more other cancer therapies (e.g., chemotherapy, radiotherapy, or surgical tumor resection), and the subject's cancer has relapsed. In certain embodiments, the subject and/or subject in need thereof has previously undergone one or more other cancer therapies (e.g., chemotherapy, radiotherapy, or stem cell transplantation), and the subject has developed resistance to the one or more other cancer therapies. In certain embodiments, the subject and/or subject in need thereof is in remission (e.g., in partial remission or in complete remission of cancer). In certain embodiments, the subject and/or subject in need thereof is refractory to one or more other cancer therapies (e.g., chemotherapy, radiotherapy, or stem cell transplantation).

In other embodiments, contemplated herein is treating a subject that is at risk of developing cancer that may (or is likely to) benefit from any treatment regimen described herein in accordance with the therapeutic methods and uses of the disclosure. A subject at risk of developing a cancer is a subject that has a higher than normal probability of developing cancer. These subjects include, for instance, subjects having a genetic abnormality that has been demonstrated to be associated with a higher likelihood of developing a cancer, subjects having a familial disposition to cancer, subjects exposed to cancer causing agents (i.e., carcinogens) such as tobacco, asbestos, or other chemical toxins, and subjects previously treated for cancer and in apparent remission. The disclosure contemplates administration of PCS6422 and capecitabine as described herein, or compositions comprising the same, to subjects at risk of developing a cancer.

Genetic variants in the DPYD gene, which encodes dihydropyrimidine dehydrogenase (DPD), can result in enzymes with reduced or absent DPD enzyme activity. Without wishing to be bound by any one theory, it is understood that subjects who have at least one copy of a non-functional DPYD variant is not able to metabolize fluorouracil (e.g., 5-FU) at normal rates. Consequently, these subjects are at an increased risk of potentially life-threatening fluorouracil toxicity, such as bone marrow suppression, gastrointestinal toxicity, and in some rare instances, neurotoxicity. As such, a subject and/or subject in need thereof in accordance with technologies of the present disclosure can include subjects with at least partial DPD enzyme activity (e.g., DPYD normal metabolizers, DPYD intermediate metabolizers). DPYD normal metabolizers have fully functional DPD enzyme activity. DPYD intermediate metabolizers can have a decreased DPD enzyme activity of about 30% to about 70% compared to the normal population. In some embodiments, a subject and/or subject in need thereof in accordance with technologies of the present disclosure is a DPYD normal metabolizer. In some embodiments, a subject and/or subject in need thereof in accordance with technologies of the present disclosure is a DPYD intermediate metabolizer.

In some embodiments, a subject and/or a subject in need thereof in accordance with technologies of the present disclosure demonstrates at least about 1% of normal DPD enzyme activity, at least about 5% of normal DPD enzyme activity, at least about 10% of normal DPD enzyme activity, at least about 20% of normal DPD enzyme activity, at least about 30% of normal DPD enzyme activity, at least about 40% of normal DPD enzyme activity, at least about 50% of normal DPD enzyme activity, at least about 60% of normal DPD enzyme activity, at least about 70% of normal DPD enzyme activity, at least about 80% of normal DPD enzyme activity, at least about 90% of normal DPD enzyme activity, at least about 95% of normal DPD enzyme activity. In some embodiments, a subject and/or subject in need thereof in accordance with technologies of the present disclosure demonstrates about 100% of normal DPD enzyme activity, about 95% of normal DPD enzyme activity, about 90% of normal DPD enzyme activity, about 85% of normal DPD enzyme activity, about 80% of normal DPD enzyme activity, about 75% of normal DPD enzyme activity, about 70% of normal DPD enzyme activity, about 60% of normal DPD activity, about 50% of normal DPD enzyme activity, about 40% of normal DPD enzyme activity, about 30% of normal DPD enzyme activity, about 20% of normal DPD enzyme activity, about 10% of normal DPD enzyme activity, about 5% of normal DPD enzyme activity, or about 1% of normal DPD enzyme activity.

A therapeutically effective amount of capecitabine can be reduced for subjects who demonstrate reduced DPD enzyme activity (e.g., DPYD intermediate metabolizers). In some embodiments, a therapeutically effective amount of capecitabine is reduced by at least about 50%, including, e.g., 60%, 70%, 80%, 90%, 95% or higher.

In some embodiments, a subject and/or a subject in need thereof in accordance with technologies of the present disclosure is not a subject with absent DPD enzyme activity, also referred to herein as “DPD deficient” (e.g., 0% DPD enzyme activity or a DPD enzyme activity below the limit of quantification). In some embodiments, a subject and/or a subject in need thereof in accordance with technologies of the present disclosure is a subject with absent DPD enzyme activity (DPD deficient).

A plurality of methods to assess DPD enzyme activity are available and known to those of ordinary skill in the art and can be utilized to determine if a subject and/or a subject in need thereof is a DPYD normal metabolizer, a DPYD intermediate metabolizer, or DPD deficient. For example, and without limitation, DPD enzyme activity can be assessed in peripheral blood mononuclear cells (PBMCs) by measuring concentrations of uracil and thymine (endogenous substrates of DPD) using reverse-phase high-performance liquid chromatography with electrospray tandem mass spectrometry. See, e.g., Henricks L M et al., Int J Cancer. 2018 Jan. 15; 142 (2): 424-430.

DPD enzyme activity of a particular subject and/or subject in need thereof may be determined based on the presence or absence of particular variants in the DYPD gene associated with a certain degree of DPD enzyme activity. A plurality of methods to determine the presence or absence of particular variants in the DYPD gene of a subject and/or a subject in need thereof are readily available and known to those of ordinary skill in the art and include, for example, sanger sequencing and next-generation sequencing. Examples of particular DYPD variants and their associated DPD enzyme activity are shown in Table 1.

TABLE 1
Activity status of selected DYPD variants

| DPD | |
|---|---|
| enzyme | |
| activity | Variants |
| Normal | No detected variant (*1), c.1627G > A (*5, |
| | rs1801159), c.85T > C (*9A, rs1801265), c.1601G > |
| | A (*4, rs1801158), c.2194G > A (*6, rs1801160), |
| | c.1003G > T (*11, rs72549306), c.2657G > A (*9B, |
| | rs1801267), 496A > G (rs2297595) |
| Intermediate | c.2846A > T (rs67376798), 1129-5923C > G and |
| | 1236G > A (HapB3), c.557A > G (rs115232898) |
| No function | c.1905 + 1G > A (*2A, rs3918290), c.1898delC (*3, |
| | rs72549303), c.295_298delTCAT (*7, rs72549309), |
| | c.703C > T (*8, rs1801266), c.2983G > T (*10, |
| | rs1801268), c.1156G > T (*12), c.1679T > G (*13, |
| | rs55886062) |

In some embodiments, a subject and/or a subject in need thereof in accordance with technologies of the present disclosure is not a subject with one or more of significantly decreased renal function, a current brain metastasis, a prolonged QT corrected for heart rate of >480 milliseconds, a history of prolonged QTc interval, ventricular trachycardia/fibrillation, or significant ventricular arrhythmia, or Torsades de Pointes, or a history of ventricular ablation for arrhythmia, history or presence of clinically significant abnormal 12-lead ECG results, congenital long QT syndrome or a family history of long QT syndrome, other significant cardiac disease (e.g., uncontrolled angina, myocardial ischemia or infarction within 6 months, congestive heart failure>Class II per the New York Heart Association, or history of myocarditis), an electrolyte disturbance (e.g., uncorrected hypokalemia/hyperkalemia, hypomagnesemia, or hypocalcemia), a known hypersensitivity to either of PCS6422 and/or capecitabine, and/or is pregnant or lactating.

### Characterization

Subjects and/or subjects of in need thereof in accordance with technologies of the present disclosure may be assessed for one or more of adverse events (e.g., any untoward medical occurrence in a subject and/or a subject in need thereof), durable clinical benefit, favorable response, long-term benefit, objective response, objective response rate, and/or progression free survival. Technologies of the present disclosure may provide one or more of a decrease in adverse events and/or an increase in durable clinical benefit, favorable response, long-term benefit, objective response, objective response rate, and/or progression free survival relative to an appropriate reference (e.g., a subject and/or subject in need thereof administered a therapeutic regimen of PCS6422 and capecitabine not described herein, a subject and/or a subject in need thereof administered a therapeutic regimen of PCS6422 and capecitabine without personalization as described herein).

### Examples of Therapeutic Regimens

The foregoing days and/or timeframes of administration, doses, drug-free periods, and/or pharmacokinetic analyses are not so limited by any particular combination and any of the above-described embodiments may be utilized in any combination. The following therapeutic regimens describe particular examples of combinations of the foregoing embodiments.

In one embodiment, a therapeutic regimen of the present disclosure comprises administration of one dose of an effective amount of PCS6422 on Day 1 of the treatment cycle, administration of one dose of a therapeutically effective amount of capecitabine on Day 2 of the treatment cycle, and a drug-free period on Days 3 and 4 of the treatment cycle. In some embodiments, the treatment cycle is repeated (e.g., based on the results of pharmacokinetic analyses as described herein).

In one embodiment, a therapeutic regimen of the present disclosure comprises administration of a single 40 mg dose of PCS6422 on Day 1 (i.e., in the morning) of the treatment cycle, administration twice daily of divided doses of 50-450 mg/day of capecitabine on each of Days 2-6 of the treatment cycle, and a drug-free period on Days 7-14 of the treatment cycle. In some embodiments, the treatment cycle is repeated (e.g., based on the results of pharmacokinetic analyses as described herein).

In one embodiment, a therapeutic regimen of the present disclosure comprises administration of a single 40 mg dose of PCS6422 on Day 1 (i.e., in the morning) of the treatment cycle, administration twice daily of divided doses of 50-450 mg/day of capecitabine on each of Days 2-3 of the treatment cycle, a drug-free period on Days 4-5 of the treatment cycle, administration of a single 40 mg dose of PCS6422 on Day 6 (i.e., in the morning) of the treatment cycle, administration twice daily of divided doses of 50-450 mg/day of capecitabine on each of Days 7-8 of the treatment cycle, and a drug-free period on Days 9-14 of the treatment cycle. In some embodiments, the treatment cycle is repeated (e.g., based on the results of pharmacokinetic analyses described herein).

In one embodiment, a therapeutic regimen of a first treatment cycle of the present disclosure comprises administration of a single 40 mg dose of PCS6422 on Day 1 (i.e., in the morning) of the treatment cycle, administration twice daily of divided doses of 50-450 mg/day of capecitabine on each of Days 2-8 of the treatment cycle, and a drug-free period on Days 9-14 of the treatment cycle. On Days 3-9 of the first treatment cycle, a blood sample obtained from the subject was measured for one or more of: α-fluoro-β-alanine (FBAL), 5′-deoxy-5-fluorocytidine (5′-DFCR), doxifluridine (5′-DFUR), capecitabine, and 5-fluorouracil (5-FU) and a ratio of measured FBAL to measured 5-FU (FBAL: 5-FU ratio) was determined. Based on one or more of the measured 5-FU, the measured FBAL, and/or the FBAL: 5-FU ratio, the total days of dosing of capecitabine is adjusted in a second treatment cycle to when PCS6422 no longer has an effect on potency.

In one embodiment, a therapeutic regimen of a first treatment cycle of the present disclosure comprises administration of a single 40 mg dose of PCS6422 on Day 1 (i.e., in the morning) of the treatment cycle, administration twice daily of divided doses of 50-450 mg/day of capecitabine on each of Days 2-4 of the treatment cycle, and a drug-free period on Days 5-7 of the treatment cycle. On Days 3-4 of the first treatment cycle, a blood sample obtained from the subject was measured for one or more of: α-fluoro-β-alanine (FBAL), 5′-deoxy-5-fluorocytidine (5′-DFCR), doxifluridine (5′-DFUR), capecitabine, and 5-fluorouracil (5-FU) and a ratio of measured FBAL to measured 5-FU (FBAL: 5-FU ratio) was determined. Based on the one or more of the measured 5-FU, the measured FBAL, and/or the FBAL: 5-FU ratio, the total days of dosing of capecitabine is adjusted in a second treatment cycle to when PCS6422 no longer has an effect, or has a minimal effect, on potency, while maintaining at least 3 days without capecitabine administration (including the day of PCS6422 administration of the second treatment cycle) and a total of a 7 day cycle. In some embodiments, every third treatment cycle is replaced with a drug-free period of 7 days.

In one embodiment, a therapeutic regimen of a treatment cycle of the present disclosure comprises administration of a single 40 mg dose of PCS6422 on Day 1 (i.e., in the morning) of the treatment cycle, administration twice daily of divided doses of 50-450 mg/day of capecitabine on each of Days 2-4 of the treatment cycle. On each of Days 2-4 of the treatment cycle, a blood sample obtained from the subject was measured for one or more of: α-fluoro-β-alanine (FBAL), 5′-deoxy-5-fluorocytidine (5′-DFCR), doxifluridine (5′-DFUR), capecitabine, and 5-fluorouracil (5-FU) and a ratio of measured FBAL to measured 5-FU (FBAL: 5-FU ratio) was determined. Based on the measured 5-FU, the measured FBAL, or the FBAL: 5-FU ratio, additional administration of twice daily divided doses of 50-450 mg/day of capecitabine would be continued or stopped. If the measured 5-FU, the measured FBAL, or the FBAL: 5-FU ratio, were less than a certain 5-FU threshold, greater than or equal to a certain FBAL threshold, or greater than or equal to a certain FBAL: 5-FU ratio, respectively, administration of additional doses of capecitabine is stopped and a drug-free period is instituted. In some embodiments, after the drug-free period, steps (i) and (ii) are repeated.

In one embodiment, a therapeutic regimen of a treatment cycle of the present disclosure comprises administration of a single 40 mg dose of PCS6422 on Day 1 (i.e., in the morning) of the treatment cycle, administration twice daily of divided doses of 50-450 mg/day of capecitabine on each of Days 2-15 of the treatment cycle, as long as blood samples obtained from the subject have a measured 5-FU greater than or equal to 2.0 ng/ml, a measured FBAL less than 15 ng/mL, or FBAL: 5-FU ratio less than 7.5. When blood samples obtained from the subject have a measured 5-FU less than 2.0 ng/mL, FBAL greater than or equal to 15 ng/ml, or FBAL: 5-FU ratio greater than or equal to 7.5, administration of additional doses of the therapeutically effective amount of capecitabine is stopped and a drug-free period commences, at least until the measured level of 5-FU is greater or equal to than 2.0 ng/ml and FBAL is less than 15 ng/ml for 2-14 days.

In one embodiment, a therapeutic regimen of a treatment cycle of the present disclosure comprises administration of an effective dose of PCS6422 on Day 1 (e.g., as a single dose or a divided dose) of the treatment cycle, administration of a therapeutically effective amount of capecitabine on each of Days 2-8 of the treatment cycle (e.g., as a single dose or a divided dose), and a drug-free period on Days 9-14 of the treatment cycle. In some embodiments, the treatment cycle is repeated (e.g., based on the results of pharmacokinetic analyses or a therapeutic index comprising such pharmacokinetic analyses as described herein). In some embodiments, based on the measured 5-FU, the measured FBAL, and/or the FBAL: 5-FU ratio, or the therapeutic index, additional administration of capecitabine would be continued or stopped.

In one embodiment, a therapeutic regimen of a treatment cycle of the present disclosure comprises administration of an effective dose of PCS6422 on Day 1 (e.g., as a single dose or a divided dose) of the treatment cycle, administration of a therapeutically effective amount of capecitabine on each of Days 2-4 of the treatment cycle (e.g., as a single dose or a divided dose), and a drug-free period on Days 5-14 of the treatment cycle. In some embodiments, the treatment cycle is repeated (e.g., based on the results of pharmacokinetic analyses or a therapeutic index comprising such pharmacokinetic analyses as described herein). In some embodiments, based on the measured 5-FU, the measured FBAL, and/or the FBAL: 5-FU ratio, or the therapeutic index, additional administration of capecitabine would be continued or stopped.

In one embodiment, a therapeutic regimen of a treatment cycle of the present disclosure comprises administration of an effective dose of PCS6422 on Day 1 (e.g., as a single dose or a divided dose) of the treatment cycle, administration of a therapeutically effective amount of capecitabine on each of Days 2-3 of the treatment cycle (e.g., as a single dose or a divided dose), and a drug-free period on Days 4-7 of the treatment cycle. In some embodiments, the treatment cycle is repeated (e.g., based on the results of pharmacokinetic analyses or a therapeutic index comprising such pharmacokinetic analyses as described herein). In some embodiments, based on the measured 5-FU, the measured FBAL, and/or the FBAL: 5-FU ratio, or the therapeutic index, additional administration of capecitabine would be continued or stopped.

In one embodiment, a therapeutic regimen of a treatment cycle of the present disclosure comprises administration of an effective dose of PCS6422 on Day 1 (e.g., as a single dose or a divided dose) of the treatment cycle, administration of a therapeutically effective amount of capecitabine on each of Days 2-4 of the treatment cycle (e.g., as a single dose or a divided dose), administration of a second effective dose of PCS6422 on Day 5 (e.g., as a single dose or a divided dose) of the treatment cycle, administration of a therapeutically effective amount of capecitabine on each of Days 6-8 of the treatment cycle (e.g., as a single dose or a divided dose), and a drug-free period on Days 9-14 of the treatment cycle. In some embodiments, the treatment cycle is repeated (e.g., based on the results of pharmacokinetic analyses or a therapeutic index comprising such pharmacokinetic analyses as described herein). In some embodiments, based on the measured 5-FU, the measured FBAL, and/or the FBAL: 5-FU ratio, or the therapeutic index, additional administration of capecitabine would be continued or stopped.

In one embodiment, a therapeutic regimen of a treatment cycle of the present disclosure comprises administration of an effective dose of PCS6422 on Day 1 (e.g., as a single dose or a divided dose) of the treatment cycle, administration of a therapeutically effective amount of capecitabine on each of Days 2-8 of the treatment cycle (e.g., as a single dose or a divided dose), administration of a second effective dose of PCS6422 on Day 5 (e.g., as a single dose or a divided dose) of the treatment cycle, and a drug-free period on Days 9-14 of the treatment cycle. In some embodiments, the treatment cycle is repeated (e.g., based on the results of pharmacokinetic analyses or a therapeutic index comprising such pharmacokinetic analyses as described herein). In some embodiments, based on the measured 5-FU, the measured FBAL, and/or the FBAL: 5-FU ratio, or the therapeutic index, additional administration of capecitabine would be continued or stopped.

In one embodiment, a therapeutic regimen of a treatment cycle of the present disclosure comprises administration of an effective dose of PCS6422 on Day 1 (e.g., as a single dose or a divided dose) of the treatment cycle, administration of a therapeutically effective amount of capecitabine on each of Days 2-15 of the treatment cycle (e.g., as a single dose or a divided dose), and a drug-free period on Days 16-21 of the treatment cycle. In some embodiments, the treatment cycle is repeated (e.g., based on the results of pharmacokinetic analyses or a therapeutic index comprising such pharmacokinetic analyses as described herein). In some embodiments, based on the measured 5-FU, the measured FBAL, and/or the FBAL: 5-FU ratio, or the therapeutic index, additional administration of capecitabine would be continued or stopped.

In one embodiment, a therapeutic regimen of a treatment cycle of the present disclosure comprises administration of an effective dose of PCS6422 on Day 1 (e.g., as a single dose or a divided dose) of the treatment cycle, administration of a therapeutically effective amount of capecitabine on each of Days 2-3 of the treatment cycle (e.g., as a single dose or a divided dose), a drug-free period on Days 4-7 of the treatment cycle, administration of a second effective dose of PCS6422 on Day 8 (e.g., as a single dose or a divided dose) of the treatment cycle, administration of a therapeutically effective amount of capecitabine on each of Days 9-10 of the treatment cycle (e.g., as a single dose or a divided dose), a drug-free period on Days 11-14 of the treatment cycle, administration of a third effective dose of PCS6422 on Day 15 (e.g., as a single dose or a divided dose) of the treatment cycle, administration of a therapeutically effective amount of capecitabine on each of Days 16-17 of the treatment cycle (e.g., as a single dose or a divided dose), and drug-free period on Days 18-28 of the treatment cycle. In some embodiments, the treatment cycle is repeated (e.g., based on the results of pharmacokinetic analyses or a therapeutic index comprising such pharmacokinetic analyses as described herein). In some embodiments, based on the measured 5-FU, the measured FBAL, and/or the FBAL: 5-FU ratio, or the therapeutic index, additional administration of capecitabine would be continued or stopped.

### EXAMPLES

### Example 1: Cohorts 1 and 2A-PCS6422 and Capecitabine 14-Day Cycle

Subjects with advanced, metastatic or unresectable gastrointestinal (GI) tract tumors that are refractory or intolerant to existing available therapies are administered with a combination of PCS6422 and capecitabine in a 14-day cycle. 40 mg of PCS6422 is administered to the subjects on day 1. Subsequently, 75 mg of capecitabine is administered to the subjects daily (Cohort 1) or twice daily (Cohort 2A, morning and night) on days 2-8 followed by 6 days of a drug-free period (days 9-14).

Blood samples for pharmacokinetic (PK) analysis of PCS6422 and its major metabolite, 5-acetyluracil, are drawn in cycle 1 on day 1 at 0 (pre-dose), 1, 2, 3, 5, 7, 9, and 24 hours post PCS6422 administration. On the first day of capecitabine treatment (Cycle 1, day 2), blood samples for PK analysis of capecitabine, 5-fluorouracil (5-FU), and the quantifiable main metabolites, α-fluoro-β-alanine (FBAL), 5′-deoxy-5-fluorocytidine (5′-DFCR), and doxifluridine (5′-DFUR) are obtained at the following time points relative to administration of the first dose of capecitabine (dose 1): 0 (pre-dose/trough), 0.5, 1, 2, 3, 5, 7, and at 9 hours. In cycle 1 on Day 8, blood samples are obtained at the following time points relative to administration of the dose of capecitabine (dose 7 of cycle 1): 0 (pre-dose/trough), 0.5, 1, 2, 3, 5, 7, 9, 24, 30, and 48 hours post-capecitabine dose. A capecitabine blood sample is also drawn on cycle 2, day 1, prior to the dose of PCS6422.

Blood samples are also drawn for the analysis of DPD activity using uracil and dihydrouracil concentrations at screening and at the following times during Cycle 1: Day 1, Day 2, Day 8, Day 9, and Day 10. A blood sample is also drawn on Cycle 2, day 1 prior to the dose of PCS6422. All blood samples for analysis of uracil and dihydrouracil concentrations are taken prior a subject's meal, if possible. Actual sample time is recorded. Validated analytical methods are used to determine the concentrations of PCS6422, 5-acetyluracil, capecitabine, 5-FU, FBAL, 5′-DFCR, and 5′-DFUR, uracil, and/or dihydrouracil in plasma.

Exemplary PK results are shown in Table 1.1.

TABLE 1.1
Examples of results from 5-FU and FBAL analysis in a Cohort 1 (C1) and 2A (C2A).

| Time | | 5FU D 2 0-9 | | | | | 5FU D 8 | 5FU D 8 0-9 | | |
|---|---|---|---|---|---|---|---|---|---|---|
| from | | hrs (ng/ml) | | 5FU D 3 | 5FU D 4 | 5FU D 5 | Cmin | hrs (ng/ml) | | 5FU D 9 |
| D 2 | | Cp | Cp | (ng/ml) | (ng/ml) | (ng/ml) | (ng/ml) | Cp | Cp | (ng/ml) |
| Cap | Pt | Low | High | 24 | 48 | 72 | 144 | Low | High | 168 |
| C1 | 1 | 85.6 | 309.0 | NA | NA | NA | <2 ng/ml | <2 ng/ml | 7.94 | <2 ng/ml |
| C2A | 2 | 164.0 | 450.0 | NA | NA | NA | <2 ng/ml | <2 ng/ml | 21.6 | <2 ng/ml |
| C2A | 3 | 77.5 | 323.0 | NA | NA | NA | <2 ng/ml | <2 ng/ml | 9.41 | <2 ng/ml |
| C2A | 4 | 8.1 | 466.0 | NA | NA | NA | <2 ng/ml | <2 ng/ml | 6.47 | <2 ng/ml |

| Time | | FBAL D 2 0-9 | | | | | FBAL D 8 | FBAL D 8 0-9 | | |
|---|---|---|---|---|---|---|---|---|---|---|
| from | | hrs (ng/ml) | | FBAL D 3 | FBAL D 4 | FBAL D 5 | Cmin | hrs (ng/ml) | | FBAL D 9 |
| D 2 | | Cp | Cp | (ng/ml) | (ng/ml) | (ng/ml) | (ng/ml) | Cp | Cp | (ng/ml) |
| Cap | Pt | Low | High | 24 | 48 | 72 | 144 | Low | High | 168 |
| C1 | 1 | <15 ng/ml | <15 ng/ml | NA | NA | NA | <15 ng/ml | 25.4 | 139.0 | <15 ng/ml |
| C2A | 2 | <15 ng/ml | <15 ng/ml | NA | NA | NA | 22.4 | 33.0 | 235.0 | 21.9 |
| C2A | 3 | <15 ng/ml | <15 ng/ml | NA | NA | NA | 20.2 | 20.5 | 204.0 | 27.8 |
| C2A | 4 | 22.6 | 26.0 | NA | NA | NA | 52 | 44.0 | 206.0 | 35.4 |

| | | | | | | | Ratio | Ratio | | |
|---|---|---|---|---|---|---|---|---|---|---|
| | | Ratio | | Ratio | Ratio | Ratio | (FBAL: | (FBAL: | | Ratio |
| Time | | (FBAL: | | (FBAL: | (FBAL: | (FBAL: | 5-FU) | 5-FU) | | (FBAL: |
| from | | 5-FU) | | 5-FU) | 5-FU) | 5-FU) | D 8 | D 8 0-9 hrs | | 5-FU) |
| D 2 | | Cp | Cp | D 3 | D 4 | D 5 | Cmin | Cp | Cp | D 9 |
| Cap | Pt | Low | High | 24 | 48 | 72 | 144 | Low | High | 168 |
| C1 | 1 | 0.18 | 0.05 | NA | NA | NA | 7.50 | 12.70 | 17.51 | 7.50 |
| C2A | 2 | 0.09 | 0.03 | NA | NA | NA | 11.20 | 16.50 | 10.88 | 10.95 |
| C2A | 3 | 0.19 | 0.05 | NA | NA | NA | 10.10 | 10.25 | 21.68 | 13.90 |
| C2A | 4 | 2.80 | 0.06 | NA | NA | NA | 26.00 | 22.00 | 31.84 | 17.70 |

NA indicates not applicable because no sample was obtained or analyzed.

Fully active DPD (e.g., DPD that is not inhibited by PCS6422) is represented by data on Day 8 in subjects that receive PCS6422 on day 1 (C1, C2A). On Day 2, DPD is inhibited, but de novo formation of DPD is also observed. The lowest and highest 5-FU plasma concentrations (Cp) during 0-9 hours following capecitabine dosing ranges from 8.1 ng/ml-164 ng/ml (Cp low) and 309 ng/mL-466 ng/ml (Cp high). FBAL Cp high on Day 2 (0-9 hours post capecitabine dose) ranges from less than 15 ng/mL to 26 ng/ml and Cp low on Day 2 (0-9 hours post capecitabine dose) ranges from less than 15 ng/mL to 22.6 ng/mL.

### Example 2: Cohorts 2B and 2D-PCS6422 and Capecitabine 14-Day Cycle

Subjects with advanced, metastatic or unresectable gastrointestinal (GI) tract tumors that are refractory or intolerant to existing available therapies were administered with a combination of PCS6422 and capecitabine in a 14-day cycle. 40 mg of PCS6422 was administered to the subjects on both of day 1 and day 5 (Cohort 2B) or on only day 1 (Cohort 2D). Subsequently, 75 mg of capecitabine was administered to the subjects twice daily (morning and night) on days 2-8 followed by 6 days of a drug-free period (days 9-14).

Blood samples for PK analysis of PCS6422 and its major metabolite, 5-acetyluracil in Cycle 1 on Day 1 was drawn at 0 (pre-dose), 1, 2, 3, 5, 7, 9 and 24 hours. The 24-hour post dose sample was collected prior to capecitabine administration on Day 2.

On the first day of capecitabine treatment (Cycle 1, Day 2), blood samples for PK analysis of capecitabine, 5-fluorouracil (5-FU), and the quantifiable main metabolites, α-fluoro-β-alanine (FBAL), 5′-deoxy-5-fluorocytidine (5′-DFCR), and doxifluridine (5′-DFUR) were obtained at the following time points relative to administration of the first dose of capecitabine (Day 1): 0 (pre-dose), 0.5, 1, 2, 3, 5, 7, and 9 hours. In Cycle 1 on Day 8, blood samples were obtained at the following time points relative to administration of the morning dose of capecitabine at time 0 (pre-dose/trough), 0.5, 1, 2, 3, 5, 7, and 9 hours.

Additional PK blood samples for capecitabine and its main metabolites were obtained prior to administration of the morning dose of capecitabine on Days 3, 4, 5, 6, 7, 9. Validated analytical methods were used to determine the concentrations of PCS6422, 5-aceyluracil, capecitabine, 5-FU, FBAL, 5′-DFCR, and 5′-DFUR in plasma.

Exemplary PK results are shown in Table 2.1.

TABLE 2.1
Results from 5-FU and FBAL analysis in a Cohort 2B (C2B) and 2D (C2D).

| | | 5FU D 2 | | | | | | 5FU D 8 | | |
|---|---|---|---|---|---|---|---|---|---|---|
| | | 0-9 hrs | | | | | 5FU D 8 | 0-9 hrs | | |
| Time | | (ng/ml) | | 5FU D 3 | 5FU D 4 | 5FU D 5 | Cmin | (ng/ml) | | 5FU D 9 |
| from | | Cp | Cp | (ng/ml) | (ng/ml) | (ng/ml) | (ng/ml) | Cp | Cp | (ng/ml) |
| D 2 Cap | Pt | Low | High | 24 | 48 | 72 | 144 | Low | High | 168 |
| C2B | 102-105 | 70.6 | 546.0 | 18.9 | <2 ng/ml | 2.8 | 15.7 | 8.91 | 403 | 5.46 |
| C2B | 106-106 | 6.4 | 197.0 | 27.0 | <2 ng/ml | 3.3 | 15.5 | 13.6 | 166 | 4.27 |
| C2B (Dose | 103-107 | 81.6 | 574.0 | 579.0 | 606.0 | 364.0 | 880.0 | 780 | 1090 | 439 |
| Limiting | | | | | | | | | | |
| Toxicity) | | | | | | | | | | |
| C2B DLT | 104-108 | 283.0 | 637.0 | 653.0 | 5.7 | <2 ng/ml | 149.0 | 112 | 713 | 53.8 |
| C2B | 106-109 | 11.8 | 542.0 | 4.0 | 6.8 | 270.0 | 50.9 | 37.2 | 424 | 8.27 |
| C2D | 105-110 | 12.8 | 312.0 | 13.4 | 5.6 | <2 ng/ml | <2 ng/ml | <2 ng/ml | 100 | <2 ng/ml |
| C2D | 104-111 | 218.0 | 746.0 | <2 ng/ml | 8.4 | 110.0 | <2 ng/ml | 2.64 | 57.7 | <2 ng/ml |

| | | FBAL D 2 | | | | | | FBAL D 8 | | |
|---|---|---|---|---|---|---|---|---|---|---|
| | | 0-9 hrs | | | | | FBAL D 8 | 0-9 hrs | | |
| Time | | (ng/ml) | | FBAL D 3 | FBAL D 4 | FBAL D 5 | Cmin | (ng/ml) | | FBAL D 9 |
| from | | Cp | Cp | (ng/ml) | (ng/ml) | (ng/ml) | (ng/ml) | Cp | Cp | (ng/ml) |
| D 2 Cap | Pt | Low | High | 24 | 48 | 72 | 144 | Low | High | 168 |
| C2B | 102-105 | 24.4 | 33.1 | 45.9 | 17.8 | 20.5 | 32.1 | 33.4 | 144.0 | 17.10 |
| C2B | 106-106 | <15 ng/ml | <15 ng/ml | <15 ng/ml | <15 ng/ml | <15 ng/ml | 41.5 | 34.5 | 90.7 | 52.30 |
| C2B DLT | 103-107 | <15 ng/ml | <15 ng/ml | <15 ng/ml | 87.2 | 207.0 | 101.0 | 93.6 | 151.0 | 241.00 |
| C2B DLT | 104-108 | <15 ng/ml | <15 ng/ml | 87.0 | 58.8 | 55.1 | 109.0 | 89.4 | 148.0 | 80.30 |
| C2B | 106-109 | 15.1 | 28.9 | <15 ng/ml | 65.0 | 30.4 | 51.7 | 49.5 | 136.0 | 23.10 |
| C2D | 105-110 | 18.3 | 22.6 | 33.3 | 51.8 | 53.6 | 40.8 | 40.9 | 170.0 | 50.6 |
| C2D | 104-111 | <15 ng/ml | <15 ng/ml | 73.6 | 109.0 | 64.7 | 186.0 | 175.0 | 463.0 | 82.7 |

| | | | | | | | | Ratio | | |
|---|---|---|---|---|---|---|---|---|---|---|
| | | | | | | | Ratio | (FBAL: | | |
| | | Ratio | | Ratio | Ratio | Ratio | (FBAL: | 5-FU) | | Ratio |
| | | (FBAL: | | (FBAL: | (FBAL: | (FBAL: | 5-FU) | D 8 | | (FBAL: |
| Time | | 5-FU) | | 5-FU) | 5-FU) | 5-FU) | D 8 | 0-9 hrs | | 5-FU) |
| from | | Cp | Cp | D 3 | D 4 | D 5 | Cmin | Cp | Cp | D 9 |
| D 2 Cap | Pt | Low | High | 24 | 48 | 72 | 144 | Low | High | 168 |
| C2B | 102-105 | 0.35 | 0.06 | 2.43 | 8.90 | 7.24 | 2.04 | 3.75 | 0.36 | 3.13 |
| C2B | 106-106 | 2.33 | 0.08 | 0.56 | 7.50 | 4.52 | 2.68 | 2.54 | 0.55 | 12.25 |
| C2B DLT | 103-107 | 0.18 | 0.03 | 0.03 | 0.14 | 0.57 | 0.11 | 0.12 | 0.14 | 0.55 |
| C2B DLT | 104-108 | 0.05 | 0.02 | 0.13 | 10.32 | 27.55 | 0.73 | 0.80 | 0.21 | 1.49 |
| C2B | 106-109 | 1.28 | 0.05 | 3.78 | 9.52 | 0.11 | 1.02 | 1.33 | 0.32 | 2.79 |
| C2D | 105-110 | 1.43 | 0.07 | 2.49 | 9.25 | 26.80 | 20.40 | 20.45 | 1.70 | 25.30 |
| C2D | 104-111 | 0.07 | 0.02 | 36.80 | 13.04 | 0.59 | 93.00 | 66.29 | 8.02 | 41.35 |

NA indicates not applicable because no sample was obtained or analyzed.

Fully active DPD (e.g., DPD that is not inhibited by PCS6422) is represented by data on Day 8 in subjects that received PCS6422 on day 1 (C2D). On Day 2, DPD was inhibited, but de novo formation of DPD was also observed. The lowest and highest 5-FU plasma concentrations (Cp) during 0-9 hours following capecitabine dosing ranged from 6.4 ng/ml-283 ng/ml (Cp low) and 197 ng/mL-746 ng/ml (Cp high). The 5-FU Cp for patients with dose-limiting toxicity at 24 hours after the Day 2 morning dose was 579 ng/mL and 653 ng/ml for patients 103-107 and 104-108, respectively, while for other patients 5-FU was observed at less than 2 ng/ml-27.0 ng/mL. FBAL Cp high on Day 2 (0-9 hours post capecitabine dose) ranged from less than 15 ng/ml to 33.1 ng/mL.

What is claimed is:

1. A method of determining frequency of PCS6422 and capecitabine administration to a subject comprising:
(i) administering at least one dose of an effective amount of PCS6422 to the subject;
(ii) about 12 to about 24 hours after administering the at least one dose of the effective amount of PCS6422, administering at least one dose of a therapeutically effective amount of capecitabine to the subject
wherein a blood sample obtained from the subject was measured for α-fluoro-β-alanine (FBAL) and 5-fluorouracil (5-FU);
(iii) determining one or more of the measured 5-FU, the measured FBAL, and/or a ratio of measured FBAL to measured 5-FU (FBAL: 5-FU ratio); and
(iv) after a drug-free period, repeating steps (i) and (ii) when at least one of the following conditions are met: the measured 5-FU was less than a certain 5-FU threshold, the measured FBAL is greater than or equal to a certain FBAL threshold, the FBAL: 5-FU ratio determined was greater than or equal to a certain FBAL: 5-FU ratio threshold, the measured 5-FU is zero, or the measured FBAL is zero.

2. The method of claim 1, wherein the therapeutically effective amount of capecitabine is administered to the subject about 12 hours after administering the effective amount of PCS6422.

3. The method of claim 1, wherein the therapeutically effective amount of capecitabine is administered to the subject about 24 hours after administering the effective amount of PCS6422.

4. The method of claim 1, wherein the therapeutically effective amount of capecitabine is administered to the subject at both of about 12 hours and about 24 hours after administering the effective amount of PCS6422.

5. The method of claim 1, wherein the blood sample obtained from the subject was obtained about 12 to about 24 hours after administering the therapeutically effective amount of capecitabine to the subject.

6. The method of claim 1, wherein FBAL and 5-FU were measured daily starting on the first day of capecitabine treatment.

7. The method of claim 1, wherein steps (i) and (ii) are repeated 1-7, 8 or 14 days after step (ii).

8. A method of determining frequency of capecitabine administration to a subject comprising:
(i) administering at least one dose of an effective amount of PCS6422 to the subject;
(ii) about 12 to about 24 hours after administering the at least one dose of the effective amount of PCS6422, administering at least one dose of a therapeutically effective amount of capecitabine to the subject, wherein a blood sample obtained from the subject was measured for α-fluoro-β-alanine (FBAL) and/or 5-fluorouracil (5-FU);
(iii) determining one or more of the measured 5-FU, the measured FBAL, or a ratio of measured FBAL to measured 5-FU (FBAL: 5-FU ratio); and
(iv) stopping administering an additional dose of the therapeutically effective amount of capecitabine to the subject when the measured 5-FU was less than a certain 5-FU threshold, the measured FBAL is greater than or equal to a certain FBAL threshold, or the FBAL: 5-FU ratio determined was greater than or equal to a certain FBAL: 5-FU ratio threshold.

9. The method of claim 8, wherein stopping administering an additional dose of the therapeutically effective amount of capecitabine to the subject is followed by a drug-free period.

10. The method of claim 9, wherein after the drug-free period, steps (i) and (ii) are repeated.

11. A method of treating cancer in a subject in need thereof comprising:
(i) administering at least one dose of an effective amount of PCS6422 to the subject;
(ii) administering at least one dose of a therapeutically effective amount of capecitabine to the subject about 12 to about 24 hours after administering the at least one dose of the effective amount of PCS6422;
(iii) measuring metabolites comprising one or more of α-fluoro-β-alanine (FBAL), 5′-deoxy-5-fluorocytidine (5′-DFCR), doxifluridine (5′-DFUR), and 5-fluorouracil (5-FU) in a blood sample obtained from the subject; and
(iv) determining whether the therapeutic regimen should be adjusted based on a therapeutic index comprising the metabolite measurements determined in step (iii).
